(19) United States
(12) Patent Application Publication
(10) Pub. No.: US 2022/0133702 A1
(43) Pub. Date: May 5, 2022

(54) CYSTIC FIBROSIS TRANSMEMBRANE CONDUCTANCE REGULATOR MODULATORS FOR TREATING AUTOSOMAL DOMINANT POLYCYSTIC KIDNEY DISEASE

(71) Applicant: The Johns Hopkins University, Baltimore, MD (US)

(72) Inventor: Liudmila Cebotaru, Baltimore, MD (US)

(21) Appl. No.: 17/513,060

(22) Filed: Oct. 28, 2021

Related U.S. Application Data
(60) Provisional application No. 63/107,796, filed on Oct. 30, 2020.

(51) Int. Cl. A61K 31/443; A61P 13/12

(52) U.S. Cl. CPC A61K 31/443; A61P 13/12

(57) ABSTRACT

Described are methods of treating cystic kidney disease. Also disclosed are methods of reducing the size and/or number of cysts in autosomal dominant polycystic kidney disease.

### TECHNICAL FIELD

The present disclosure relates to the use of cystic fibrosis transmembrane conductance regulator modulators to treat cystic kidney disease.

### BACKGROUND

Autosomal dominant polycystic kidney disease (ADPKD) is one of the most prevalent, potentially lethal, monogenic human disorders, i.e., a disorder caused by a defect or defects two genes (pkd1 and pkd2). ADPKD is associated with large interfamilial and intrafamilial variability, which can be explained, in part, by its genetic heterogeneity and modifier genes. ADPKD also is the most common of the inherited cystic kidney diseases, a group of disorders having related, but distinct pathogenesis, and which are characterized by the development of renal cysts and various extrarenal manifestations. In the case of ADPKD, these manifestations include cysts in other organs, such as the liver, seminal vesicles, pancreas, and arachnoid membrane, as well as other abnormalities, such as intracranial aneurysms and dolichoectasias, aortic root dilatation aneurysms, mitral valve prolapse, and abdominal wall hernias. More than 50% of patients afflicted with ADPKD eventually develop end stage kidney disease and require dialysis or kidney transplantation. ADPKD is estimated to affect at least 1 in every 1000 individuals worldwide.

### SUMMARY

The presently disclosed subject matter, in part, identifies CFTR modulators as a potential therapeutic target for treating autosomal dominant polycystic kidney disease (ADPKD).

In some aspects, the presently disclosed subject matter provides a method of treating cystic kidney disease in a subject in need thereof, the method comprising administering a cystic fibrosis transmembrane conductance regulator (CFTR) modulator to the subject. In certain aspects, the cystic kidney disease is autosomal dominant polycystic disease.

In some aspects of the presently disclosed methods, the cystic fibrosis transmembrane conductance regulator (CFTR) modulator reduces kidney cysts size and/or number. In other aspects, cAMP concentration is reduced in the kidney of the subject compared to a kidney of a reference subject not administered the CFTR

modulator. In yet other aspects, Hsp27 is decreased in the kidney of the subject compared to a kidney of a reference subject not administered the CFTR modulator. In even yet other aspects, Hsp90 is decreased in the kidney of the subject compared to a kidney of a reference subject not administered the CFTR modulator. In other aspects, Hsp70 is decreased in the kidney of the subject compared to a kidney of a reference subject not administered the CFTR modulator. In certain aspects, chloride level is reduced in a cyst lumen. In other aspects, water is reduced in a cyst lumen.

Certain aspects of the presently disclosed subject matter having been stated hereinabove, which are addressed in whole or in part by the presently disclosed subject matter, other aspects will become evident as the description proceeds when taken in connection with the accompanying Examples and Figures as best described herein below.

### BRIEF DESCRIPTION OF THE FIGURES

The patent or application file contains at least one drawing executed in color. Copies of this patent or patent application publication with color drawings will be provided by the Office upon request and payment of the necessary fee.

Having thus described the presently disclosed subject matter in general terms, reference will now be made to the accompanying Figures, which are not necessarily drawn to scale, and wherein:

FIG. 1A, FIG. 1B, FIG. 1C, FIG. 1D, FIG. 1E, FIG. 1F, and FIG. 1G show that a representative CFTR modulator, e.g., VX-809, slows cyst growth and improves renal function in pkd1−/− mice. (FIG. 1A) Representative images of postnatal day (PND) 21 kidney sections from DMSO- and VX-809-treated mice. Significant reductions occurred in (FIG. 1B) cyst index, (FIG. 1C) kidney weight, and (FIG. 1D) kidney-to-body weight ratio. (FIG. 1E) No differences were noted in body weight. Also reduced was (FIG. 1F) blood urea nitrogen (BUN) and (FIG. 1G) creatinine levels. Methods: The total kidney area and total cystic area were measured with ImageJ (provided by NIH). Cystic index=100 X (total cystic area/total kidney area) and is expressed as a percentage. Columns represent means±standard error (SEM) for DMSO (vehicle)-treated (n=4-5) and VX-809 mice. *P<0.05; **P<0.01 (for all graphs). Statistical analysis was performed using an unpaired two-tailed Student's t-test;

FIG. 2A, FIG. 2B, FIG. 2C, and FIG. 2D show (FIG. 2A-FIG. 2B) VX-809 inhibits cyst growth in the presence of forskolin. Cells were treated with DMSO (control), VX-809, or C18 (10 μM) plus or minus forskolin on Days 0, 2, 4, 6, 8, 10, 12, 14. (FIG. 2C-FIG. 2D): VX-809 reduces the size of established cysts in the presence of forskolin. Cells were treated with DMSO (control), C18 or VX-809 (10 μM) from 9-16 (7) days. All pictures were taken on Day 16. Columns represent means±SEM (n=6). The average cyst size from the control group was considered 100%, and the sizes of the rest of the cysts were compared with this average. ****P<0.0001. Cyst size was estimated from the cross-sectional area;

FIG. 3A, FIG. 3B, FIG. 3C, and FIG. 3D show cell proliferation is reduced by VX-809. (FIG. 3A-FIG. 3B) Proliferation in kidneys of Pkd1^{fl/fl}; Pax8^{rtTA}; TetO-cre mice. Representative images of Ki67 (a cellular marker for proliferation) staining of PN21 kidney sections from DMSO- and VX-809-treated mice. Arrows indicate Ki67-positive cells. Pictures were acquired with a Zeiss microscope equipped with 20× objective. (FIG. 3C) Summary data for Ki67-positive cells. Columns represent averages±standard errors of DMSO (vehicle)-treated (n=3) and VX-809-treated (n=3) mice. Statistical analysis was performed using a two-tailed Student's t test. (FIG. 3D) Proliferation in PN cells. PN cells were treated with VX-809 or DMSO). The bromodeoxyuridine (BrdU) concentration in the cells was measured by using a BrdU cell proliferation assay kit (Millipore Sigma#2750) according to the manufacturer's protocol. Columns represent averages±SEs of the OD of BrdU at 450/550 nM. Data were analyzed using Student's t-test, with n=5-7. Methods were described previously (62). *P>0.05; **P<0.01;

FIG. 4A, FIG. 4B, FIG. 4C, FIG. 4D, and FIG. 4E show (FIG. 4A-FIG. 4B) cAMP levels are reduced by VX-809. (FIG. 4A) cAMP levels in mouse tissue were reduced by VX-809. The kidney samples (n=6) analyzed were taken from those depicted in FIG. 1. (FIG. 4B) Confluent PN and PH cells were treated with VX-809 (10 μM) or DMSO for 16 hand then treated with forskolin (100 μM) for 30 min before the cells were harvested for the assay. Columns represent means±SEM. Statistical analysis was performed using a two-tailed Student's t-test. Each set of data is from three individual wells; Note that resting cAMP is greater in PN vs PH cells as previously shown (25). Also note that forskolin caused a large increase in cAMP. A smaller increase occurred with IBMX. However, the increase induced by forskolin alone was similar to that by IBMX plus forskolin. VX-809 treatment reduced the levels of cAMP when compared to untreated PN cells, in either the presence or absence of forskolin. (FIG. 4C, FIG. 4D, and FIG. 4E) Adenylyl cyclase expression. (FIG. 4C) Western blot showing the expression of adenylyl cyclase (AC) 6 and AC3 in treated and control PN cells. (FIG. 4D-FIG. 4E) Columns represent means±SEM of the AC3 and AC6 expression. The data were analyzed by non-parametric t-test. The experiment was repeated four times. For all graphs, *P<0.05, **P<0.01, and ***P<0.001;

FIG. 5A, FIG. 5B, FIG. 5C, FIG. 5D, FIG. 5E, and FIG. 5F show intracellular Ca²⁺ (F340/F380) levels obtained by ratiometric Fura-2 AM analysis of PN cells treated with VX-809 (10 μM) for 16 h. (FIG. 5A) Representative traces of intracellular Ca²⁺ release in response to ATP (100 μM) in PN cells and cells treated with VX-809 (10 μM). (FIG. 5B, FIG. 5C) Graphs summarizing resting calcium levels (FIG. 5B) and the average amplitude of Ca²⁺ release (FIG. 5C) in response to ATP. (FIG. 5D) Representative traces of ER Ca²⁺ release in response to thapsigargin (4 μM) in PN cells treated with VX-809. (FIG. 5E, FIG. 5F) Resting calcium levels (FIG. 5E) and the average amplitude of Ca²⁺ release (FIG. 5F) in response to thapsigargin. Amplitude was measured as the standard deviation of the signal base to peak Δf/f. Significance between the two groups was analyzed using Student's t-test, n=4-5). *P<0.05, ***P<0.001. The data show that VX-809 reduces ER Ca²⁺ release;

FIG. 6A and FIG. 6B show PC2 expression is unchanged by VX-809. (FIG. 6A) Western blot showing expression of PC2 in treated or control cells. (FIG. 6B) Columns represent the means±SEM of the PC2 expression. The data were analyzed by non-parametric t-test. The experiment was repeated six (control) or seven times;

FIG. 7A, FIG. 7B, FIG. 7C, FIG. 7D, FIG. 7E, and FIG. 7F show chaperone expression is altered in mice by VX-809. (FIG. 7A-FIG.7B) Representative Western blot images of HSP27, 70, 90 and HSP40 in lysates of kidney tissue from no cyst induced (ND), cyst induced with Doxycycline (D) or cyst induced pkd−/− mice treated with VX-809 (D+VX-809) 30 mg/kg BW. (FIG. 7C, FIG. 7D, FIG. 7E, and FIG. 7F). Columns represent averages±standard errors of HSP27, 70, 90 and HSP40 expression. Data were analyzed by non-parametric t-test. n=4 for each treated and control groups. Black lines between lanes represent representative experiments from the same gel. *P<0.05; **P<0.01;

FIG. 8A, FIG. 8B, FIG. 8C, FIG. 8D, FIG. 8E, FIG. 8F, FIG. 8G, and FIG. 8H show chaperone expression is altered in PH and PN cells by VX-809. (FIG. 5A) Western blot showing expression of HSP27, HSP70 and HSP90 in VX-809 treated or control PN cells. (FIG. 8B, FIG. 8C, and FIG. 8D) Columns represent averages±standard errors of the HSP27, HSP70 and HSP90 expression and show that VX-809 reduces the expression of HSP27, HSP70 and HSP90. (FIG. 8E). Western blot of HSP27, HSP70 and HSP90 in PH vs. PN cells. (FIG. 5F, FIG. 5G, and FIG. 8H) Columns represent averages±standard errors of the HSP27, HSP70 and HSP90 expression and show that the expression of HSP 27 and 90 are higher in PN vs. PH cells. Whereas, Hsp70 is higher in PH vs. PN cells. Data were analyzed by non-parametric t test. Experiment was repeated 4-5 times. Cells were grown in 10-cm culture dishes at permissive conditions (33° C.) with y-interferon in culture media. Cells were then transferred to non-permissive conditions at 37° C., y-interferon free culture media and evaluated at full confluence. At day four, cells were treated with VX-809 (10 μM) for 16 h and harvested on fifth day for assay. *P<0.05., **P<0.01, ***P<0.001, ****P<0.0001;

FIG. 9A, FIG. 9B, FIG. 9C, FIG. 9D, FIG. 9E, FIG. 9F, FIG. 9G, and FIG. 9H show Hsp27, HSp70, and Hsp90 expression in PN cells treated with VX-809 and/or cycloheximide. (A) Pkd1 null (PN) cells were treated with VX-809 (10 μM) and/or with cycloheximide (25 μM) for indicated time points before being harvested for the assay. (FIG. 9A) Western blot showing expression of Hsp27, Hsp70, and Hsp90 in VX-809-(16 h), cycloheximide-treated, or control PN cells. ((FIG. 9B. (FIG. 9C, and (FIG. 9D) Columns represent averages±standard errors of Hsp27, Hsp70, and Hsp90 expression in cells treated with VX-809 and/or cycloheximide. ((FIG. 9E) Western blot showing expression of Hsp27, Hsp70, and Hsp90 in PN cells treated with VX-809+cycloheximide or control PN cells. ((FIG. 9F, FIG. 9G, and FIG. 9H) Columns represent averages±standard errors of Hsp27, Hsp70, and Hsp90 expression in cells treated only with cycloheximide or in control PN cells. Data were analyzed by non-parametric t-test. The experiments were repeated 3-5 times. *P<0.05, **P<0.01;

FIG. 10 shows apoptosis is reduced by VX-809. Apoptosis was measured using the EnzChek Caspase-3 Assay Kit (Invitrogen #E13184). Cells were treated either with VX-809 or with DMSO at indicated concentrations for 16 h or left untreated (control). Both treated and control cells were then harvested, lysed, and assayed as described in the manufacturer's protocol. Reactions were carried out at room temperature, and fluorescence was measured in a fluorescence microplate reader (SpectraMax M3), with excitation at 496 nm and emission detection at 520 nm. Background fluorescence, determined for a no-enzyme control, has been subtracted from each value. Ac-DEVD-CHO was used as an inhibitor control (Inh-cntr1) to confirm the correlation between signal detection and caspase3-like protease activity. Data were analyzed using one-way and ANOVA multiple comparisons. n=4-5. ****P<0.0001;

FIG. 11A. FIG. 11B, FIG. 11C, FIG. 11D, and FIG. 11E show expression of ER stress markers GRP78, Ero1, and GADD in Pkd1-1 mice treated with VX-809. (FIG. 11A & FIG. 11D) Representative Western blot images of GRP78, ErO1, and GADD in lysates of kidney tissue from no cyst induced (ND), cyst induced with doxycycline (D) or cyst induced pkd−/− mice treated with VX-809 (D+VX-809) 30 mg/kg BW. (FIG. 11B, FIG. 11C and FIG. 11E) Columns represent averages±standard errors of GRP78, Ero1, and GADD153 expression. Data were analyzed by non-parametric t-test. n=4 for each treated and control groups. Black lines between lanes represent representative experiments from the same gel but loaded in different lanes. **P<0.01, ***P<0.001;

FIG. 12A, FIG. 12B, and FIG. 12C show GADD153 expression in kidneys of Pkd1fllfl; Pax8^{rtTA}; TetO-cre mice. Immunofluorescence images representing the GADD expression (green) in DMSO (FIG. 12A) or VX-809 (FIG. 12B) treated PN21 Pkd1^{fl/fl} mouse. Columns (FIG. 12C) represent % area of GADD immunopositive (green) cells in DMSO or VX-809 treated PN21 Pkd1^{fl/fl} mouse kidney sections. Data are expressed as mean±1 SEM of DMSO (vehicle)-treated (n=4) and VX-809-treated (n=4) mice. Statistical analysis was performed using a 2-tailed Student t-test. DMSO, dimethyl sulfoxide. Kidneys were fixed in 4% paraformaldehyde as described (62). Used here was the GADD153 antibody (Sc-7351), goat antirabbit (A21429, AlexaFluor 555, 1:1,000; Life Technologies, Carlsbad, Calif.) and DAPI (H-1200; Vector Laboratories, Burlingame, Calif.). Pictures were acquired with a Zeiss microscope equipped a 20× objective. Cells positive for GADD153 and the total number of cells were measured with ImageJ. The results are expressed as percentages;

FIG.13A and FIG. 13B show a schematic representation of a proposed mechanism of action of VX-809 on cyst growth. (FIG. 13A) Gene profiling of human cysts shows an increase in HSF1 expression as compared to normal kidneys (63), HSF1 activation leads to the transcriptional up-regulation of several HSPs which most likely drives the increase in heat shock factors noted here and in other studies (16). It was shown (25) previously that thapsigargin-induced Ca²⁺ release from the ER is enhanced in PN cells vs PH cells leading to an increase in cAMP via adenylyl cyclase 3. Thapsigargin inhibits the SERCA pump (sarcolemma-endoplasmic reticulum Ca²⁺ pump) (64) causing Ca²⁺ to leak out of the ER via the IP3R (inositol triphosphate receptor). The combination of elevated cAMP, enhanced release of Ca²⁺ from the ER and increased heat shock factor expression fuels cyst growth. (FIG. 13B) One of the factors that upregulates HSF1 is aberrant Ca²⁺ regulation (65). Thus, VX-809 reduces Hsp27 expression either directly (15) or via a reduction in thapsigargin induced ER Ca²⁺ release. Inhibiting thapsigargin-induced Ca²⁺ release is associated with reduced cAMP via calmodulin regulation of AC3. Thus, VX-809 by inhibiting thapsigargin induced ER Ca²⁺ release, reducing heat shock proteins and cAMP robs the cyst of several components that fuel cyst growth;

FIG. 14A and FIG. 14B show NHE3 expression in PN cells treated with VX-809. NHE3 expression in PN cells treated with VX-809 is significantly increased compared with PN cells;

FIG. 15A and FIG. 15B show NHE3 activity in PN and PH cells (FIG. 15A and FIG. 15B) and NHE3 activity in PN cells treated with VX-809. NHE3 activity in PN cells is significantly reduced compared with the control PH cells; NHE3 activity is significantly increased in PN cells treated with VX-809 compared with PN cells;

FIG. 16A, FIG. 16B, and FIG. 16C show NHE activity in PN/PH cells or PN cells treated with VX-809;

FIG. 17A and FIG. 17B show images of confocal microscopy localization studies of PC2 and localization markers in control and treated cells (FIG. 17A) and graphs showing Pearson's correlation coefficient for PC2 and localization markers in control and VX-809 treated cells (FIG. 17B). These studies show that there is a statistically significant move of PC2 out of the ER to the Golgi;

FIG. 18A and FIG. 18B show images of confocal microscopy localization studies of CFTR and localization markers in control and treated cells (FIG. 18A) and graphs showing Pearson's correlation coefficient for CFTR and localization markers in control and VX-809 treated cells (FIG. 18B). These studies show that CFTR moves significantly out from the ER to basolateral and apical membrane;

FIG. 19A, FIG. 19B, FIG. 19C, and FIG. 19D show CFTR expression in PN cells treated with VX-809 (10 μM) for 16 h. VX-809 treatment enhanced the CFTR expression compared with control PN cells;

FIG. 20 shows confocal microscopic images of stained kidney sections in Pkd1 mice;

FIG. 21 is a graph of the percentage of cells with ciliary staining;

FIG. 22 shows an immunoblot and graph of PC2 protein levels in kidney tissue of Pkd1^{fl/fl}; Pax8^{rtTA}; TetO-cre mice; and

FIG. 23 shows an immunoblot and graph of PC2 protein levels in kidney tissue of wild-type and Pkd11^{RC/RC} mice.

### DETAILED DESCRIPTION

The presently disclosed subject matter now will be described more fully hereinafter with reference to the accompanying Figures, in which some, but not all embodiments of the presently disclosed subject matter are shown. Like numbers refer to like elements throughout. The presently disclosed subject matter may be embodied in many different forms and should not be construed as limited to the embodiments set forth herein; rather, these embodiments are provided so that this disclosure will satisfy applicable legal requirements. Indeed, many modifications and other embodiments of the presently disclosed subject matter set forth herein will come to mind to one skilled in the art to which the presently disclosed subject matter pertains having the benefit of the teachings presented in the foregoing descriptions and the associated Figures. Therefore, it is to be understood that the presently disclosed subject matter is not to be limited to the specific embodiments disclosed and that modifications and other embodiments are intended to be included within the scope of the appended claims.

Disclosed herein are modulators and methods for the treatment of cystic kidney disease. The cystic kidney disease may be autosomal dominant kidney disease. Methods of treating cystic kidney disease are disclosed. The methods may comprise reducing the number and/or size of kidney cysts.

### I. Method Of Treating Cystic Kidney Disease

The disclosed modulators may be used in methods for treatment of cystic kidney disease. The methods of treatment may comprise administering to a subject in need of such treatment a composition comprising a therapeutically effective amount of the modulators disclosed herein. Treatment of such cystic kidney diseases, by administering modulators of this disclosure, may be administered alone or in combination with another active agent as part of a therapeutic regimen to a subject in need thereof. Specifically, the methods of treatment disclosed herein may treat autosomal dominant polycystic kidney disease.

a. Cystic Kidney Diseases

The disclosed modulators and methods may be used to treat cystic kidney disease. The disclosed modulators and methods may reduce the size and/or number of kidney cysts. Cystic kidney disease may cause cysts to form on one or both the kidneys. Cystic kidney disease may cause cysts to form in one or both kidneys. Kidney cysts may contain fluid. Cysts may contain solid material. Cysts may contain fluid and solid material. One kidney cyst may be present. Many kidney cysts may be present.

Symptoms of cystic kidney disease may include, but are not limited to, renal colic, back pain, flank pain, upper abdominal pain, recurrent urinary tract infections, blood in the urine, headache, fever, chills, upper abdominal swelling, kidney stones, hypertension, frequent urination, urine obstruction, reduced kidney function, and kidney failure.

Kidney cysts may be detected by ultrasound, computed tomography (CT), magnetic resonance imaging (MM), or genetic testing. Cystic kidney diseases may be hereditary. Cystic kidney disease may be nonhereditary. Cystic kidney diseases may be spontaneous. Cystic kidney disease may lead to altered kidney function. Cystic kidney disease may lead to decreased kidney function. Cystic kidney disease may lead to kidney failure.

The disclosed compositions and methods may eliminate the need for surgery to remove cysts. The classification of cystic kidney diseases that may be treated with the disclosed compositions and methods includes, but is not limited to, adult onset, pediatric onset, autosomal dominant polycystic kidney disease, autosomal recessive polycystic kidney disease, nephronophthisis, multi-cystic kidney disease, medullary sponge kidney, simple renal cysts, minimally complex renal cysts, intermediate renal cysts, clearly malignant renal cysts, Von-Hippel-Lindau disease tuberculosis sclerosis complex, localized renal cystic disease, congenital nephrosis, familial nephrotic syndrome, familial hypoplastic glomerulocystic disease, juvenile nephronophthesis-medullary cystic disease complex, juvenile nephronophthesis, acquired renal cystic disease, benign multilocular cyst, cystic nephroma, calyceal diverticulum, pyelogenic cyst, and multicystic dysplastic kidney. Cystic kidney disease may occur concurrently with another condition or disease. In some embodiments, the cystic kidney disease is autosomal dominant polycystic kidney disease.

b. Autosomal Dominant Polycystic Kidney Disease

The disclosed compositions and methods may be used to treat autosomal dominant polycystic kidney disease. Subjects with ADPKD may have multiple kidney cysts. Subjects with ADPKD may have hypertension. Subjects with ADPKD may have reduced kidney function. Subjects with ADPKD may have renal failure. ADPKD may be associated with the protein PC1. ADPKD may be associated with the protein PC2. Cysts may develop in or on the kidneys. Cysts may develop in a nephron segment. ADPKD cysts may contain fluid. ADPKD cyst fluid may be produced by a cAMP-dependent mechanism. The formation of ADPKD cysts may involve activation of cystic fibrosis transmembrane conductance regulator (CFTR). Activation of the CFTR may secrete chloride into the cyst lumen. Activation of the CFTR may lead to the accumulation of sodium into the cyst lumen. Activation of the CFTR may lead to the accumulation of water into the cyst lumen.

c. Cystic Fibrosis Transmembrane Conductance Regulator

The disclosed modulators and methods may target the cystic fibrosis transmembrane conductance regulator (CFTR). CFTR is a member of the ATP binding cassette family. CFTR may function as a cAMP-dependent chloride channel. Channel activation may be mediated by cycles of regulatory domain phosphorylation, ATP-binding by the nucleotide-binding domains, and ATP hydrolysis. Mutations in the CFTR gene cause cystic fibrosis. The most frequently occurring mutation in cystic fibrosis, DeltaF508, results in impaired folding and trafficking of the encoded protein. CFTR may line the luminal membrane of ADPKD cysts. CFTR may contribute to cAMP-dependent fluid secretion and cyst growth in ADPKD. Modulators may be used to target CFTR.

d. Cystic Fibrosis Transmembrane Conductance Regulator Modulators

In one embodiment, disclosed are cystic fibrosis transmembrane conductance regulator (CFTR) modulators. The CFTR modulator may be a small molecule. A modulator may be potentiator. A potentiator may activate a channel. Representative potentiators include, but are not limited to, ivacaftor (VX-770). A modulator may be a corrector. A corrector may affect protein folding. A modulator may be an amplifier. An amplifier may increase gene expression. Generally, a CFTR amplifier enhances the effect of a potentiator or corrector. Examples of CFTR amplifiers are PTI 130 and PTI-428. Examples of amplifiers also are disclosed in WO2015138909 and WO2015138934, each of which is incorporated by reference in its entirety. The 20 presently disclosed methods also can include a CFTR stabilizer. A CFTR stabilizer can enhance the stability of corrected CFTR that has been treated with a corrector, corrector/potentiator, or CFTR modulator combinations. An example of a CFTR stabilizer is cavosonstat (N91115). Examples of stabilizers are also disclosed in WO2012048181, which is incorporated by reference in its entirety.

In some embodiments of the presently disclosed methods, the CFTR modulator is selected from the group consisting of a potentiator, a corrector, an amplifier, and combinations thereof. In particular embodiments, the CFTR modulator can be a corrector. In other embodiments, the CFTR modulator can be a potentiator. In other embodiments, the CFTR modulator can be an amplifier. In some embodiments, the CFTR modulator can include a combination of a corrector and a potentiator; a combination of a corrector and an amplifier; or a combination of a corrector, a potentiator, and an amplifier. In yet other embodiments, the presently disclosed methods can include a stabilizer in combination with a CFTR modulator, such as a potentiator, a corrector, and/or an amplifier.

Further, a modulator may alter protein trafficking. The modulator may reduce ER Ca²⁺ release. The modulator may inhibit ER Ca²⁺ release. The inhibition of ER Ca²⁺ release may prevent ADPKD cysts from responding to growth stimuli. The modulator may decrease Hsp27, Hsp90, and/or Hsp70. The decrease in Hsp27, Hsp90, and/or Hsp70 may decrease the size or number of ADPKD cysts. The modulator may decrease cAMP. The modulator may reduce cAMP levels by reducing AC3. The modulator may increase CFTR protein expression in the kidney. Increased CFTR protein expression in the kidney may lead to an increase in chloride. The modulator may prevent the secretion of chloride into the cyst lumen. The prevention of the secretion of chloride into the cyst lumen may prevent sodium and water from entering the cyst lumen. Preventing water from entering the cyst lumen may reduce the size of a cyst. Preventing water from entering the lumen of the cyst may treat ADPKD. The modulator may restore renal cells in ADPKD to a non-cyst forming phenotype, including negating the ability of cAMP to sustain and stimulate cyst growth. The modulator may lead to sodium reabsorption. The modulator may restore sodium reabsorption. The modulator may move CFTR from the ER to Basolateral and Apical Membranes. The modulator may move PC2 from the ER to the Golgi.

The modulator may reduce cyst growth in the proximal tubule (PT) of the kidney, distal tubule (DT) of the kidney, and/or the collecting duct of the kidney. The modulator may restore AQP2 in the collecting duct. The modulator may lead to sodium, chloride and water reabsorption thereby reducing cyst size by absorbing fluid from the cyst lumen Modulators may directly act on CFTR to attenuate the deleterious effects of disease. Modulators may act indirectly on CFTR to attenuate the deleterious effects of the disease.

Examples of CFTR modulators that may be used with methods disclosed herein include, but are not limited to, lumacaftor (VX-809), Corr-4a, VRT-325, C18, C4, C3, VX-770, VX-786, 4-phenylbutyrate (4PBA), VRT-532, N6022, miglustat, sildenafil and analogs thereof, ataluren (PTC124), oubain, roscovitine, suberoylanilide hydroxamic acid, latonduine and analogs thereof, SAHA, FDL169, tezacaftor (VX-661), VX-659, and VX-445. Additional potentiators and correctors are included in U.S. Pat. No. 9,981,910, which is incorporated by reference in its entirety.

e. Modes Of Administeration

Methods of treatment may include any number of modes of administering a presently disclosed modulator. Modes of administration may include tablets, pills, dragees, hard and soft gel capsules, granules, pellets, aqueous, lipid, oily or other solutions, emulsions such as oil-in-water emulsions, liposomes, aqueous or oily suspensions, syrups, elixirs, solid emulsions, solid dispersions or dispersible powders. For the preparation of pharmaceutical compositions for oral administration, the agent may be admixed with commonly known and used adjuvants and excipients such as for example, gum arabic, talcum, starch, sugars (such as, e.g., mannitose, methyl cellulose, lactose), gelatin, surface-active agents, magnesium stearate, aqueous or non-aqueous solvents, paraffin derivatives, cross-linking agents, dispersants, emulsifiers, lubricants, conserving agents, flavoring agents (e.g., ethereal oils), solubility enhancers (e.g., benzyl benzoate or benzyl alcohol) or bioavailability enhancers (e.g. Gelucire®). In the pharmaceutical composition, the agent may also be dispersed in a microparticle, e.g. a nanoparticulate composition.

For parenteral administration, the agent can be dissolved or suspended in a physiologically acceptable diluent, such as, e.g., water, buffer, oils with or without solubilizers, surface-active agents, dispersants or emulsifiers. As oils for example and without limitation, olive oil, peanut oil, cottonseed oil, soybean oil, castor oil and sesame oil may be used. More generally, for parenteral administration, the agent can be in the form of an aqueous, lipid, oily or other kind of solution or suspension or even administered in the form of liposomes or nano-suspensions.

f. Combination Therapies

The term “combination” is used in its broadest sense and means that a subject is administered at least two agents. More particularly, the term “in combination” refers to the concomitant administration of two (or more) active agents for the treatment of a, e.g., single disease state. As used herein, the active agents may be combined and administered in a single dosage form, may be administered as separate dosage forms at the same time, or may be administered as separate dosage forms that are administered alternately or sequentially on the same or separate days. In one embodiment of the presently disclosed subject matter, the active agents are combined and administered in a single dosage form In another embodiment, the active agents are administered in separate dosage forms (e.g., wherein it is desirable to vary the amount of one but not the other). The single dosage form may include additional active agents for the treatment of the disease state.

Further, the presently disclosed compositions can be administered alone or in combination with adjuvants that enhance stability of the agents, facilitate administration of pharmaceutical compositions containing them in certain embodiments, provide increased dissolution or dispersion, increase activity, provide adjuvant therapy, and the like, including other active ingredients. Advantageously, such combination therapies utilize lower dosages of the conventional therapeutics, thus avoiding possible toxicity and adverse side effects incurred when those agents are used as monotherapies.

The timing of administration of the modulators can be varied so long as the beneficial effects of the combination of these agents are achieved. Accordingly, the phrase “in combination with” refers to the administration of at least two modulators, and optionally additional agents either simultaneously, sequentially, or a combination thereof. Therefore, a subject administered a combination of at least two inhibitors, and optionally additional agents can receive at least two inhibitors, and optionally additional agents at the same time (i.e., simultaneously) or at different times (i.e., sequentially, in either order, on the same day or on different days), so long as the effect of the combination of all agents is achieved in the subject.

When administered sequentially, the agents can be administered within 1, 5, 10, 30, 60, 120, 180, 240 minutes or longer of one another. In other embodiments, agents administered sequentially, can be administered within 1, 2, 3, 4, 5, 10, 15, 20 or more days of one another. Where the agents are administered simultaneously, they can be administered to the subject as separate pharmaceutical compositions, each comprising either at least one inhibitor, and optionally additional agents, or they can be administered to a subject as a single pharmaceutical composition comprising all agents.

When administered in combination, the effective concentration of each of the agents to elicit a particular biological response may be less than the effective concentration of each agent when administered alone, thereby allowing a reduction in the dose of one or more of the agents relative to the dose that would be needed if the agent was administered as a single agent. The effects of multiple agents may, but need not be, additive or synergistic. The agents may be administered multiple times.

In some embodiments, when administered in combination, the two or more agents can have a synergistic effect. As used herein, the terms “synergy,” “synergistic,” “synergistically” and derivations thereof, such as in a “synergistic effect” or a “synergistic combination” or a “synergistic composition” refer to circumstances under which the biological activity of a combination of an agent and at least one additional therapeutic agent is greater than the sum of the biological activities of the respective agents when administered individually.

Synergy can be expressed in terms of a “Synergy Index (SI),” which generally can be determined by the method described by F. C. Kull et al. Applied Microbiology 9, 538 (1961), from the ratio determined by:

QaQA+QbQB=Synergy Index (SI)

wherein:

QA is the concentration of a component A, acting alone, which produced an end point in relation to component A;

Qa is the concentration of component A, in a mixture, which produced an end point;

QB is the concentration of a component B, acting alone, which produced an end point in relation to component B; and

Qb is the concentration of component B, in a mixture, which produced an end point.

Generally, when the sum of Qa/QA and Qb/QB is greater than one, antagonism is indicated. When the sum is equal to one, additivity is indicated. When the sum is less than one, synergism is demonstrated. The lower the SI, the greater the synergy shown by that particular mixture. Thus, a “synergistic combination” has an activity higher that what can be expected based on the observed activities of the individual components when used alone. Further, a “synergistically effective amount” of a component refers to the amount of the component necessary to elicit a synergistic effect in, for example, another therapeutic agent present in the composition.

### II. Pharmaceutical Compositions

The disclosed modulators may be incorporated into pharmaceutical compositions suitable for administration to a subject (such as a patient, which may be a human or non-human).

The pharmaceutical compositions may include a “therapeutically effective amount” or a “prophylactically effective amount” of the agent. A “therapeutically effective amount” refers to an amount effective, at dosages and for periods of time necessary, to achieve the desired therapeutic result. A therapeutically effective amount of the composition may be determined by a person skilled in the art and may vary according to factors such as the disease state, age, sex, and weight of the individual, and the ability of the composition to elicit a desired response in the individual. A therapeutically effective amount is also one in which any toxic or detrimental effects of modulators of the disclosure are outweighed by the therapeutically beneficial effects.

A “prophylactically effective amount” refers to an amount effective, at dosages and for periods of time necessary, to achieve the desired prophylactic result. Typically, since a prophylactic dose is used in subjects prior to or at an earlier stage of disease, the prophylactically effective amount may be less than the therapeutically effective amount.

For example, a therapeutically effective amount of disclosed modulators may be about 1 mg/kg to about 1000 mg/kg, about 5 mg/kg to about 950 mg/kg, about 10 mg/kg to about 900 mg/kg, about 15 mg/kg to about 850 mg/kg, about 20 mg/kg to about 800 mg/kg, about 25 mg/kg to about 750 mg/kg, about 30 mg/kg to about 700 mg/kg, about 35 mg/kg to about 650 mg/kg, about 40 mg/kg to about 600 mg/kg, about 45 mg/kg to about 550 mg/kg, about 50 mg/kg to about 500 mg/kg, about 55 mg/kg to about 450 mg/kg, about 60 mg/kg to about 400 mg/kg, about 65 mg/kg to about 350 mg/kg, about 70 mg/kg to about 300 mg/kg, about 75 mg/kg to about 250 mg/kg, about 80 mg/kg to about 200 mg/kg, about 85 mg/kg to about 150 mg/kg, and about 90 mg/kg to about 100 mg/kg.

The pharmaceutical compositions may include pharmaceutically acceptable carriers. The term “pharmaceutically acceptable carrier,” as used herein, means a non-toxic, inert solid, semi-solid or liquid filler, diluent, encapsulating material or formulation auxiliary of any type. Some examples of materials which can serve as pharmaceutically acceptable carriers are sugars such as, but not limited to, lactose, glucose and sucrose; starches such as, but not limited to, com starch and potato starch; cellulose and its derivatives such as, but not limited to, sodium carboxymethyl cellulose, ethyl cellulose and cellulose acetate; powdered tragacanth; malt; gelatin; talc; excipients such as, but not limited to, cocoa butter and suppository waxes; oils such as, but not limited to, peanut oil, cottonseed oil, safflower oil, sesame oil, olive oil, com oil and soybean oil; glycols; such as propylene glycol; esters such as, but not limited to, ethyl oleate and ethyl laurate; agar; buffering agents such as, but not limited to, magnesium hydroxide and aluminum hydroxide; alginic acid; pyrogen-free water; isotonic saline; Ringer's solution; ethyl alcohol, and phosphate buffer solutions, as well as other non-toxic compatible lubricants such as, but not limited to, sodium lauryl sulfate and magnesium stearate, as well as coloring agents, releasing agents, coating agents, sweetening, flavoring and perfuming agents, preservatives and antioxidants can also be present in the composition, according to the judgment of the formulator.

Thus, the compounds and their physiologically acceptable salts and solvates may be formulated for administration by, for example, solid dosing, eyedrop, in a topical oil-based formulation, injection, inhalation (either through the mouth or the nose), implants, or oral, buccal, parenteral, or rectal administration. Techniques and formulations may generally be found in “Remington's Pharmaceutical Sciences”, (Meade Publishing Co., Easton, Pa.). Therapeutic compositions must typically be sterile and stable under the conditions of manufacture and storage.

The route by which the disclosed modulators are administered and the form of the composition will dictate the type of carrier to be used. The composition may be in a variety of forms, suitable, for example, for systemic administration (e.g., oral, rectal, nasal, sublingual, buccal, implants, or parenteral) or topical administration (e.g., dermal, pulmonary, nasal, aural, ocular, liposome delivery systems, transdermal, or iontophoresis).

Carriers for systemic administration typically include at least one of diluents, lubricants, binders, disintegrants, colorants, flavors, sweeteners, antioxidants, preservatives, glidants, solvents, suspending agents, wetting agents, surfactants, combinations thereof, and others. All carriers are optional in the compositions.

Suitable diluents include sugars such as glucose, lactose, dextrose, and sucrose; diols such as propylene glycol; calcium carbonate; sodium carbonate; sugar alcohols, such as glycerin; mannitol; and sorbitol. The amount of diluent(s) in a systemic or topical composition is typically about 50 to about 90%.

Suitable lubricants include silica, talc, stearic acid and its magnesium salts and calcium salts, calcium sulfate; and liquid lubricants such as polyethylene glycol and vegetable oils such as peanut oil, cottonseed oil, sesame oil, olive oil, com oil and oil of theobroma. The amount of lubricant(s) in a systemic or topical composition is typically about 5 to about 10%.

Suitable binders include polyvinyl pyrrolidone; magnesium aluminum silicate; starches such as com starch and potato starch; gelatin; tragacanth; and cellulose and its derivatives, such as sodium carboxymethylcellulose, ethyl cellulose, methylcellulose, microcrystalline cellulose, and sodium carboxymethylcellulose. The amount of binder(s) in a systemic composition is typically about 5 to about 50%.

Suitable disintegrants include agar, alginic acid and the sodium salt thereof, effervescent mixtures, croscarmelose, crospovidone, sodium carboxymethyl starch, sodium starch glycolate, clays, and ion exchange resins. The amount of disintegrant(s) in a systemic or topical composition is typically about 0.1 to about 10%.

Suitable colorants include a colorant such as an FD&C dye. When used, the amount of colorant in a systemic or topical composition is typically about 0.005 to about 0.1%.

Suitable flavors include menthol, peppermint, and fruit flavors. The amount of flavor(s), when used, in a systemic or topical composition is typically about 0.1 to about 1.0%.

Suitable sweeteners include aspartame and saccharin. The amount of sweetener(s) in a systemic or topical composition is typically about 0.001 to about 1%.

Suitable antioxidants include butylated hydroxyanisole (“BHA”), butylated hydroxytoluene (“BHT”), and vitamin E. The amount of antioxidant(s) in a systemic or topical composition is typically about 0.1 to about 5%.

Suitable preservatives include benzalkonium chloride, methyl paraben and sodium benzoate. The amount of preservative(s) in a systemic or topical composition is typically about 0.01 to about 5%.

Suitable glidants include silicon dioxide. The amount of glidant(s) in a systemic or topical composition is typically about 1 to about 5%.

Suitable solvents include water, isotonic saline, ethyl oleate, glycerine, hydroxylated castor oils, alcohols such as ethanol, and phosphate buffer solutions. The amount of solvent(s) in a systemic or topical composition is typically from about 0 to about 100%.

Suitable suspending agents include AVICEL RC-591 (from FMC Corporation of Philadelphia, Pa.) and sodium alginate. The amount of suspending agent(s) in a systemic or topical composition is typically about 1 to about 8%.

Suitable surfactants include lecithin, Polysorbate 80, and sodium lauryl sulfate, and the TWEENS from Atlas Powder Company of Wilmington, Del. Suitable surfactants include those disclosed in the C.T.F.A. Cosmetic Ingredient Handbook, 1992, pp.587-592; Remington's Pharmaceutical Sciences, 15th Ed. 1975, pp. 335-337; and McCutcheon's Volume 1, Emulsifiers & Detergents, 1994, North American Edition, pp. 236-239. The amount of surfactant(s) in the systemic or topical composition is typically about 0.1% to about 5%.

Although the amounts of components in the systemic compositions may vary depending on the type of systemic composition prepared, in general, systemic compositions include 0.01% to 50% of active and 50% to 99.99% of one or more carriers. Compositions for parenteral administration typically include 0.1% to 10% of actives and 90% to 99.9% of a carrier including a diluent and a solvent.

Compositions for oral administration can have various dosage forms. For example, solid forms include tablets, capsules, granules, and bulk powders. These oral dosage forms include a safe and effective amount, usually at least about 5%, and more particularly from about 25% to about 50% of actives. The oral dosage compositions include about 50% to about 95% of carriers, and more particularly, from about 50% to about 75%.

Tablets can be compressed, tablet triturates, enteric-coated, sugar-coated, film-coated, or multiple-compressed. Tablets typically include an active component, and a carrier comprising ingredients selected from diluents, lubricants, binders, disintegrants, colorants, flavors, sweeteners, glidants, and combinations thereof. Specific diluents include calcium carbonate, sodium carbonate, mannitol, lactose and cellulose. Specific binders include starch, gelatin, and sucrose. Specific disintegrants include alginic acid and croscarmelose. Specific lubricants include magnesium stearate, stearic acid, and talc. Specific colorants are the FD&C dyes, which can be added for appearance. Chewable tablets preferably contain sweeteners such as aspartame and saccharin, or flavors such as menthol, peppermint, fruit flavors, or a combination thereof.

Capsules (including implants, time release and sustained release formulations) typically include an active compound, and a carrier including one or more diluents disclosed above in a capsule comprising gelatin. Granules typically comprise a disclosed compound, and preferably glidants such as silicon dioxide to improve flow characteristics. Implants can be of the biodegradable or the non-biodegradable type. The selection of ingredients in the carrier for oral compositions depends on secondary considerations like taste, cost, and shelf stability, which are not critical for the purposes of this invention.

Solid compositions may be coated by conventional methods, typically with pH or time-dependent coatings, such that a disclosed compound is released in the gastrointestinal tract in the vicinity of the desired application, or at various points and times to extend the desired action. The coatings typically include one or more components selected from the group consisting of cellulose acetate phthalate, polyvinyl acetate phthalate, hydroxypropyl methyl cellulose phthalate, ethyl cellulose, EUDRAGIT coatings (available from Rohm & Haas G.M.B.H. of Darmstadt, Germany), waxes and shellac.

Compositions for oral administration can have liquid forms. For example, suitable liquid forms include aqueous solutions, emulsions, suspensions, solutions reconstituted from non-effervescent granules, suspensions reconstituted from non-effervescent granules, effervescent preparations reconstituted from effervescent granules, elixirs, tinctures, syrups, and the like. Liquid compositions, which may be administered orally, may include a disclosed immunogenic proteins, compositions, and vaccines and a carrier, namely, a carrier selected from diluents, colorants, flavors, sweeteners, preservatives, solvents, suspending agents, and surfactants. Peroral liquid compositions preferably include one or more ingredients selected from colorants, flavors, and sweeteners.

Other compositions useful for attaining systemic delivery of the subject compounds include sublingual, buccal and nasal dosage forms. Such compositions typically include one or more of soluble filler substances such as diluents including sucrose, sorbitol and mannitol; and binders such as acacia, microcrystalline cellulose, carboxymethyl cellulose, and hydroxypropyl methylcellulose. Such compositions may further include lubricants, colorants, flavors, sweeteners, antioxidants, and glidants.

The disclosed modulators may be topically administered. Topical compositions that can be applied locally to the skin may be in any form including solids, solutions, oils, creams, ointments, gels, lotions, shampoos, leave-on and rinse-out hair conditioners, milks, cleansers, moisturizers, sprays, skin patches, and the like. The carrier of the topical composition preferably aids penetration of the compounds into the skin. The carrier may further include one or more optional components. Transdermal administration may be used to facilitate delivery.

The amount of the carrier employed in conjunction with a disclosed compound is sufficient to provide a practical quantity of composition for administration per unit dose of the medicament. Techniques and compositions for making dosage forms useful in the methods of this invention are described in the following references: Modern Pharmaceutics, Chapters 9 and 10, Banker & Rhodes, eds. (1979); Lieberman et al., Pharmaceutical Dosage Forms: Tablets (1981); and Ansel, Introduction to Pharmaceutical Dosage Forms, 2nd Ed., (1976).

A carrier may include a single ingredient or a combination of two or more ingredients. In the topical compositions, the carrier includes a topical carrier. Suitable topical carriers include one or more ingredients selected from phosphate buffered saline, isotonic water, deionized water, monofunctional alcohols, symmetrical alcohols, aloe vera gel, allantoin, glycerin, vitamin A and E oils, mineral oil, propylene glycol, PPG-2 myristyl propionate, dimethyl isosorbide, castor oil, combinations thereof, and the like. More particularly, carriers for skin applications include propylene glycol, dimethyl isosorbide, and water, and even more particularly, phosphate buffered saline, isotonic water, deionized water, monofunctional alcohols, and symmetrical alcohols.

The carrier of a topical composition may further include one or more ingredients selected from emollients, propellants, solvents, humectants, thickeners, powders, fragrances, pigments, and preservatives, all of which are optional.

Suitable emollients include stearyl alcohol, glyceryl monoricinoleate, glyceryl monostearate, propane-1,2-diol, butane-1,3-diol, mink oil, cetyl alcohol, isopropyl isostearate, stearic acid, isobutyl palmitate, isocetyl stearate, oleyl alcohol, isopropyl laurate, hexyl laurate, decyl oleate, octadecan-2-ol, isocetyl alcohol, cetyl palmitate, di-n-butyl sebacate, isopropyl myristate, isopropyl palmitate, isopropyl stearate, butyl stearate, polyethylene glycol, triethylene glycol, lanolin, sesame oil, coconut oil, arachis oil, castor oil, acetylated lanolin alcohols, petroleum, mineral oil, butyl myristate, isostearic acid, palmitic acid, isopropyl linoleate, lauryl lactate, myristyl lactate, decyl oleate, myristyl myristate, and combinations thereof. Specific emollients for skin include stearyl alcohol and polydimethylsiloxane. The amount of emollient(s) in a skin-based topical composition is typically about 5% to about 95%.

Suitable propellants include propane, butane, isobutane, dimethyl ether, carbon dioxide, nitrous oxide, and combinations thereof. The amount of propellant(s) in a topical composition is typically about 0% to about 95%.

Suitable solvents include water, ethyl alcohol, methylene chloride, isopropanol, castor oil, ethylene glycol monoethyl ether, diethylene glycol monobutyl ether, diethylene glycol monoethyl ether, dimethylsulfoxide, dimethyl formamide, tetrahydrofuran, and combinations thereof. Specific solvents include ethyl alcohol and homotopic alcohols. The amount of solvent(s) in a topical composition is typically about 0% to about 95%.

Suitable humectants include glycerin, sorbitol, sodium 2-pyrrolidone-5-carboxylate, soluble collagen, dibutyl phthalate, gelatin, and combinations thereof. Specific humectants include glycerin. The amount of humectant(s) in a topical composition is typically 0% to 95%.

The amount of thickener(s) in a topical composition is typically about 0% to about 95%.

Suitable powders include beta-cyclodextrins, hydroxypropyl cyclodextrins, chalk, talc, fullers earth, kaolin, starch, gums, colloidal silicon dioxide, sodium polyacrylate, tetra alkyl ammonium smectites, trialkyl aryl ammonium smectites, chemically-modified magnesium aluminum silicate, organically-modified Montmorillonite clay, hydrated aluminum silicate, fumed silica, carboxyvinyl polymer, sodium carboxymethyl cellulose, ethylene glycol monostearate, and combinations thereof. The amount of powder(s) in a topical composition is typically 0% to 95%.

The amount of fragrance in a topical composition is typically about 0% to about 0.5%, particularly, about 0.001% to about 0.1%.

Suitable pH adjusting additives include HCl or NaOH in amounts sufficient to adjust the pH of a topical pharmaceutical composition.

In an embodiment, the pharmaceutical composition may include human breast milk. The active pharmaceutical ingredient may be a component of human breast milk. The human breast milk may thus be administered to a subject in need of the active pharmaceutical ingredient.

### III. Kits

The modulators may be included in kits comprising the immunogenic proteins, compositions, and vaccines; and information, instructions, or both that use of the kit will provide treatment for medical conditions in mammals (particularly humans). The kit may include an additional pharmaceutical composition for use in combination therapy. The kit may include buffers, reagents, or other components to facilitate the mode of administration. The kit may include materials to facilitate nasal mucosal administration. The kit may include materials that facilitate sublingual administration. The information and instructions may be in the form of words, pictures, or both, and the like. In addition or in the alternative, the kit may include the medicament, a composition, or both; and information, instructions, or both, regarding methods of application of medicament, or of composition, preferably with the benefit of treating or preventing medical conditions in mammals (e.g., humans). The modulators of the invention will be better understood by reference to the following examples, which are intended as an illustration of and not a limitation upon the scope of the invention.

### IV. Definitions

Unless otherwise defined, all technical and scientific terms used herein have the same meaning as commonly understood by one of ordinary skill in the art. In case of conflict, the present document, including definitions, will control. Preferred methods and materials are described below, although methods and materials similar or equivalent to those described herein can be used in practice or testing of the present invention. All publications, patent applications, patents and other references mentioned herein are incorporated by reference in their entirety. The materials, methods, and examples disclosed herein are illustrative only and not intended to be limiting.

The modifier “about” used in connection with a quantity is inclusive of the stated value and has the meaning dictated by the context (for example, it includes at least the degree of error associated with the measurement of the particular quantity). The modifier “about” should also be considered as disclosing the range defined by the absolute values of the two endpoints. For example, the expression “from about 2 to about 4” also discloses the range “from 2 to 4.” The term “about” may refer to plus or minus 10% of the indicated number. For example, “about 10%” may indicate a range of 9% to 11%, and “about 1” may mean from 0.9-1.1. Other meanings of “about” may be apparent from the context, such as rounding off, so, for example “about 1” may also mean from 0.5 to 1.4.

The terms “administration” or “administering” as used herein may include the process in which the modulator as described herein, alone or in combination with other compounds or compositions, are delivered to a subject. The modulator may be administered in various routes including, but not limited to, oral, mucosal, mucosal nasal, parenteral (including intravenous, intra-arterial, and other appropriate parenteral routes), intrathecally, intramuscularly, subcutaneously, colonically, rectally, and nasally, transcutaneously, among others. The dosing of the modulator described herein to obtain a therapeutic or prophylactic effect may be determined by the circumstances of the subject, as known in the art. The dosing of a subject herein may be accomplished through individual or unit doses of the modulator herein or by a combined or prepackaged or pre-formulated dose of the modulator.

Administration may depend upon the amount of modulator administered, the number of doses, and duration of treatment. For example, multiple doses of the modulator may be administered. The frequency of administration of the immunogenic proteins, compositions, and vaccines may vary depending on any of a variety of factors. The duration of administration of the modulator, e.g., the period of time over which the modulator is administered, may vary, depending on any of a variety of factors, including subject response, etc.

The amount of the modulator administered may vary according to factors such as the degree of susceptibility of the individual, the age, sex, and weight of the individual, idiosyncratic responses of the individual, the dosimetry, and the like. Detectably effective amounts of the immunogenic proteins, compositions, and vaccines of the present disclosure may also vary.

As used in this specification and the appended claims, the singular forms “a”, “an”, and “the” include plural referents unless the content clearly dictates otherwise. As used in this specification and the appended claims, the term “or” is generally employed in its sense including “and/or” unless the context clearly dictates otherwise.

The terms “comprise(s),” “include(s),” “having,” “has,” “can,” “contain(s),” and variants thereof, as used herein, are intended to be open-ended transitional phrases, terms, or words that do not preclude the possibility of additional acts or structures. The singular forms “a,” “an” and “the” include plural references unless the context clearly dictates otherwise. The present disclosure also contemplates other embodiments “comprising,” “consisting of” and “consisting essentially of,” the embodiments or elements presented herein, whether explicitly set forth or not.

The term “parenterally,” as used herein, refers to modes of administration which include intravenous, intramuscular, intraperitoneal, intrastemal, subcutaneous and intraarticular injection and infusion.

A “pharmaceutically acceptable excipient,” “pharmaceutically acceptable diluent,” “pharmaceutically acceptable carrier,” or “pharmaceutically acceptable adjuvant” means an excipient, diluent, carrier, and/or adjuvant that are useful in preparing a pharmaceutical composition that are generally safe, non-toxic and neither biologically nor otherwise undesirable, and includes an excipient, diluent, carrier, and adjuvant that are acceptable for veterinary use and/or human pharmaceutical use. “A pharmaceutically acceptable excipient, diluent, carrier and/or adjuvant” as used herein includes one or more such excipients, diluents, carriers, and adjuvants.

As used herein, the term “subject,” “patient,” or “organism” includes humans and mammals (e.g., mice, rats, pigs, cats, dogs, and horses). Typical subjects to which an agent(s) of the present disclosure may be administered may include mammals, particularly primates, especially humans. For veterinary applications, suitable subjects may include, for example, livestock such as cattle, sheep, goats, cows, swine, and the like; poultry such as chickens, ducks, geese, turkeys, and the like; and domesticated animals particularly pets such as dogs and cats. For diagnostic or research applications, suitable subjects may include mammals, such as rodents (e.g., mice, rats, hamsters), rabbits, primates, and swine such as inbred pigs and the like. The subject may have cystic kidney disease. The subject may have autosomal dominant polycystic kidney disease. The subject may be at risk for developing a cystic kidney disease.

The “therapeutically effective amount” for purposes herein may be determined by such considerations as are known in the art. A therapeutically effective amount of a compound may include the amount necessary to provide a therapeutically effective result in vivo. The amount of the compound or composition must be effective to achieve a response, including but not limited to a total prevention of (e.g., protection against) of a condition, improved survival rate or more rapid recovery, improvement or elimination of symptoms associated with the condition (such as cancer), or other indicators as are selected as appropriate measures by those skilled in the art. As used herein, a suitable single dose size includes a dose that is capable of preventing or alleviating (reducing or eliminating) a symptom in a subject when administered one or more times over a suitable time period. The “therapeutically effective amount” of a compound or composition as described herein may depend on the route of administration, type of subject being treated, and the physical characteristics of the subject. These factors and their relationship to dose are well known to one of skill in the medicinal art, unless otherwise indicated.

As used herein, “treat”, “treatment”, “treating”, and the like refer to acting upon a condition with an agent to affect the condition by improving or altering it. The condition includes, but is not limited to cystic kidney disease. The cystic kidney disease may be autosomal dominant polycystic kidney disease. The aforementioned terms cover one or more treatments of a condition in a subject (e.g., a mammal, typically a human or non-human animal of veterinary interest), and include: (a) reducing the risk of occurrence of the condition in a subject determined to be predisposed to the condition but not yet diagnosed, (b) impeding the development of the condition, and/or (c) relieving the condition, e.g., causing regression of the condition and/or relieving one or more condition symptoms (e.g., treating cystic kidney disease, reducing the size and/or number of cysts).

For the recitation of numeric ranges herein, each intervening number there between with the same degree of precision is explicitly contemplated. For example, for the range of 6-9, the numbers 7 and 8 are contemplated in addition to 6 and 9, and for the range 6.0-7.0, the number 6.0, 6.1, 6.2, 6.3, 6.4, 6.5, 6.6, 6.7, 6.8, 6.9, and 7.0 are explicitly contemplated.

### EXAMPLES

### Example 1

VX-809 Reduces Cyst Growth and Improves Renal Function in Pkd1^{fl/fl}; Pax8^{fl/fl}; TetO-Cre Mice

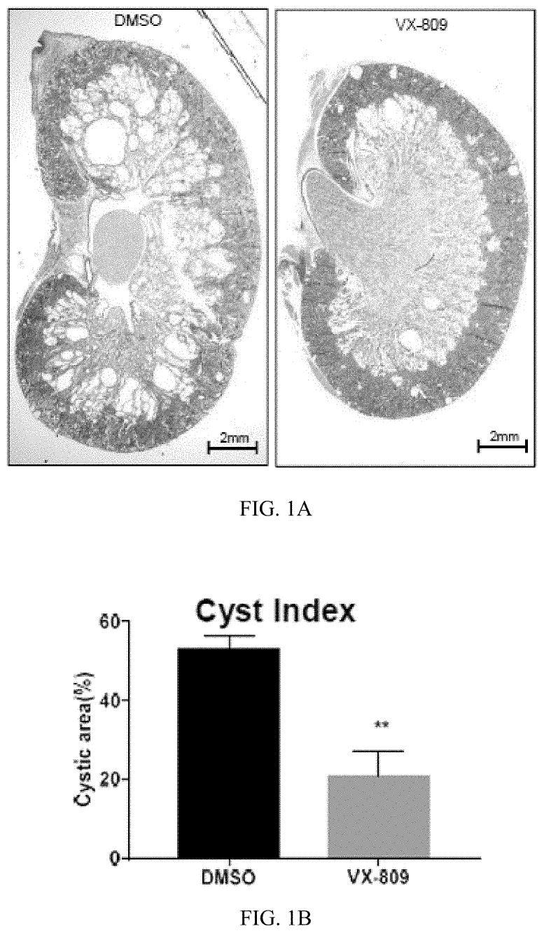

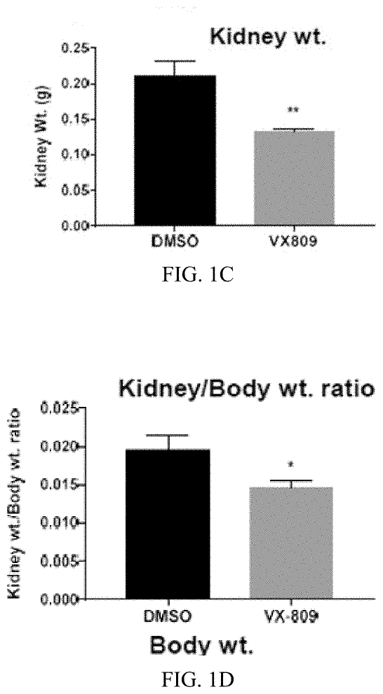

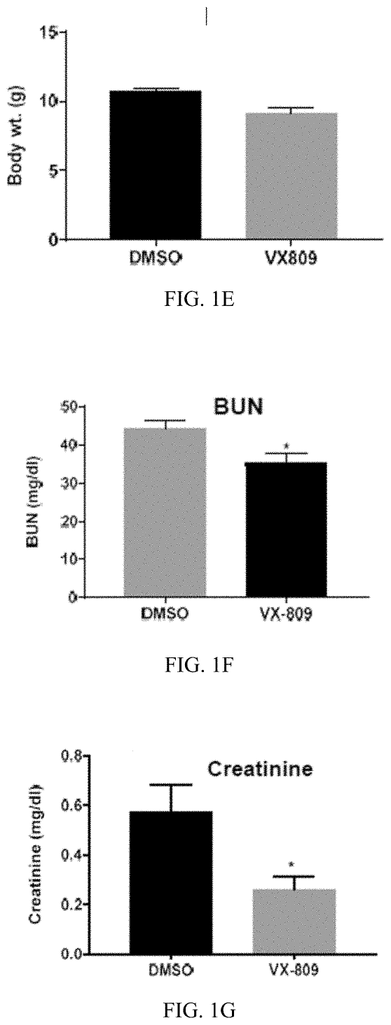

To show that VX-809 is effective in reducing cyst growth in vivo, the drug was injected into the intraperitoneal space (IP) in PkdJfllfl, Pax8^{rtTA}; TetO-cre model mice. When treated with doxycycline, these mice express Cre, causing knockout of PC1. As has previously been shown, injection of doxycycline resulted in the development of multiple large cysts in these mice at approximately 3 weeks of age (FIG. 1A). In sharp contrast, when this strain of mice was injected daily with VX-809 (30 mg/kg) or DMSO from postnatal day (PND)IO to PND20, they showed significantly less cyst growth (−60.4%) (FIG. 1A, FIG. 1B). Kidney weight (FIG. 1C) and kidney-to-body weight ratios (FIG. 1D) were also lower than those of the control mice. There was no difference in overall body weight (FIG. 1E) between the treated and untreated groups. Administration of VX-809 improved renal function, as evidenced by lowered blood urea nitrogen (BUN) (FIG. 1F) and creatinine (FIG. 1G) values in the VX-809-treated mice than in the DMSO-treated mice. The dosage of VX-809 injected was lower than the human pediatric dose, which is 500 mg of Lumacaftor per day; the adult dose is 800 mg/day.

### Example 2

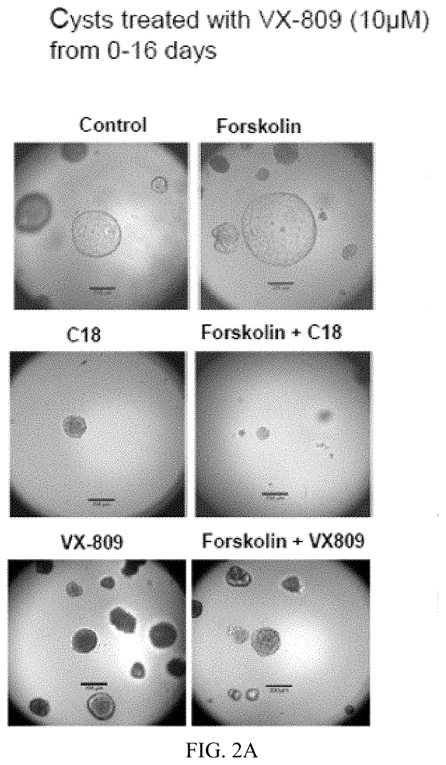

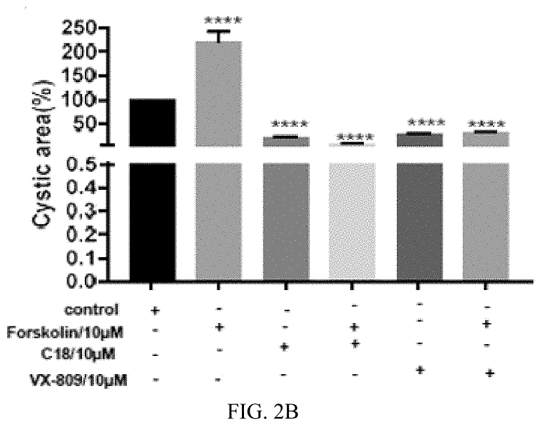

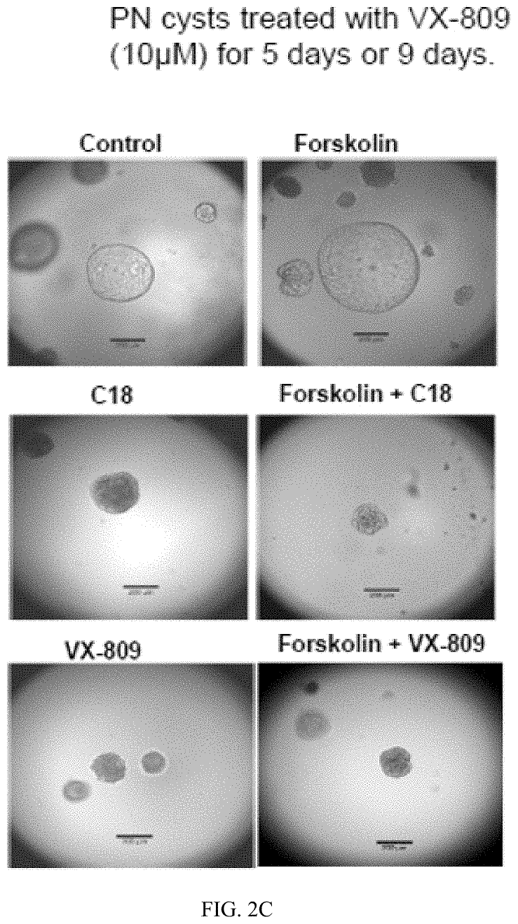

VX-809 Reduces Cyst Growth in Vitro Experiments were conducted using a model ADPKD cell line (PH=pkd1+/− heterozygote control; PN=pkd−/− knockout), clonally isolated from single parental clones obtained from a pkdf1/− mouse that was manufactured in an ImmortoMouse containing the H-2Kb-tsA58 gene. The null cells (PN) stably express the Cre recombinase. All the cells proximal tubule origin. To determine how VX-809 reduces cyst growth, cysts were grown in the presence of forskolin (FIG. 2). After treatment with forskolin, the cysts obtained were larger than those in the control cells, indicating that cyst growth is indeed cAMP-dependent, as has been shown previously. FIG. 2 shows the effect on cyst growth of C18 or VX-809 when administered every other day, as well as the same treatment administered from Days 9-16 in mice with already established cysts. This drug treatment regime was effective in dramatically reducing cyst size, even in the presence of the hyper-stimulatory environment created by forskolin. Also, VX-809 and a related compound C18 were both able to reduce cyst growth equally well in the presence and absence of forskolin (FIG. 2). This finding shows that VX-809 can overcome the powerful stimulatory effect of forskolin-induced increases in cAMP levels and still reduce cyst growth. Taken together with the animal data provided in FIG. 1, these in vitro results clearly indicate that CFTR correctors are effective in reducing cyst growth.

### Example 3

VX-809 Reduces Proliferation

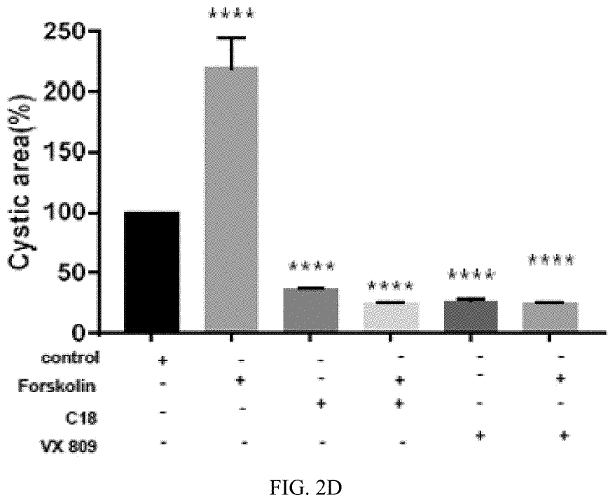

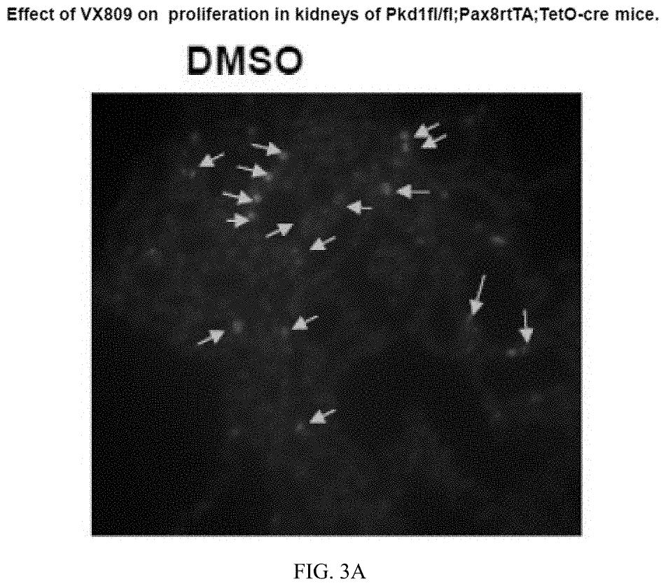

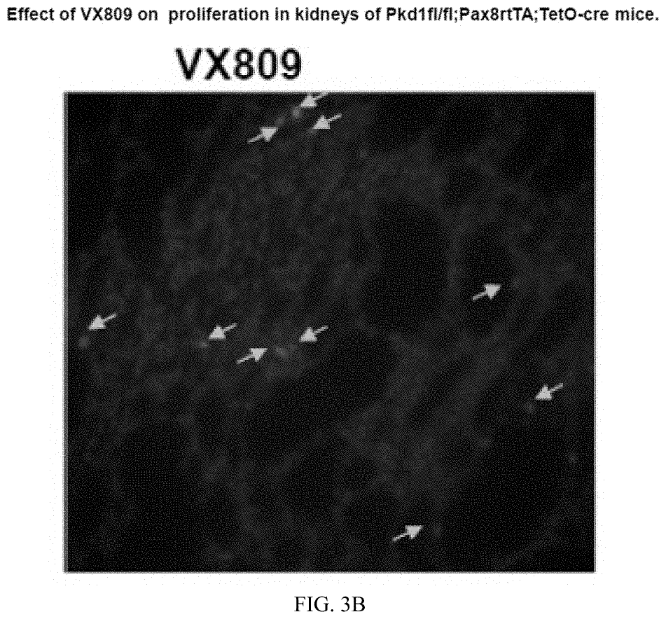

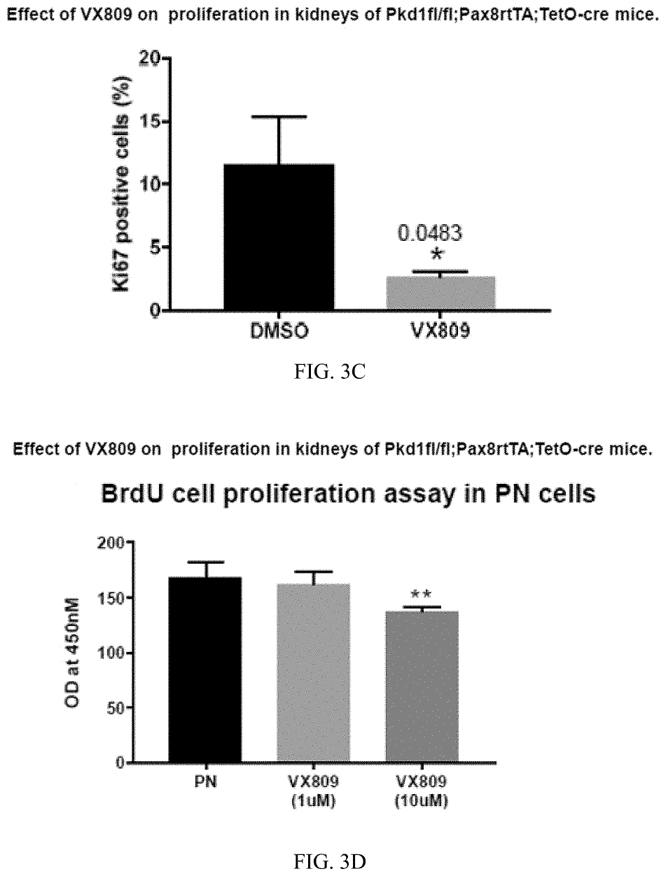

A hallmark of the cysts in ADPKD is an increase in proliferation in response to cAMP. Therefore, it was determined whether VX-809 would affect proliferation. Administration of VX-809 at 30 mg/kg (see FIG. 1) to mice (FIG. 3A-C) or to cells (FIG. 3D) at 1 and 10 μM did significantly inhibit cell proliferation when compared to that of DMSO-treated mice or cells.

### Example 4

VX-809 Downregulates cAMP Levels

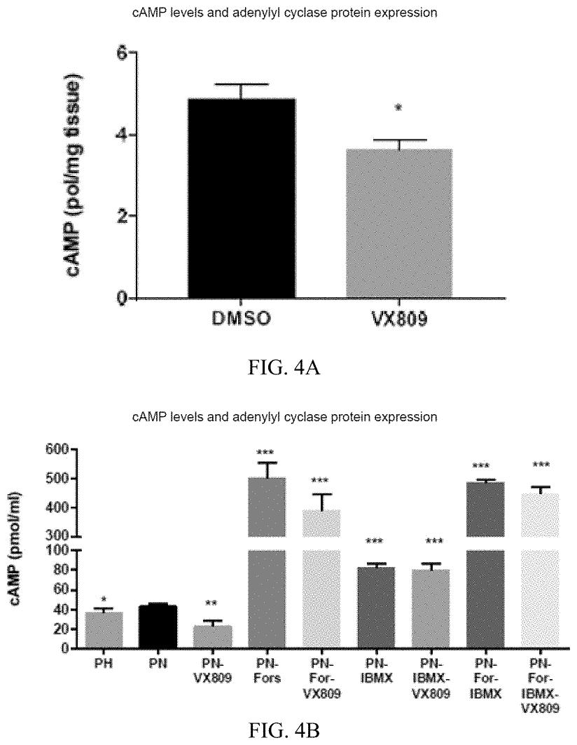

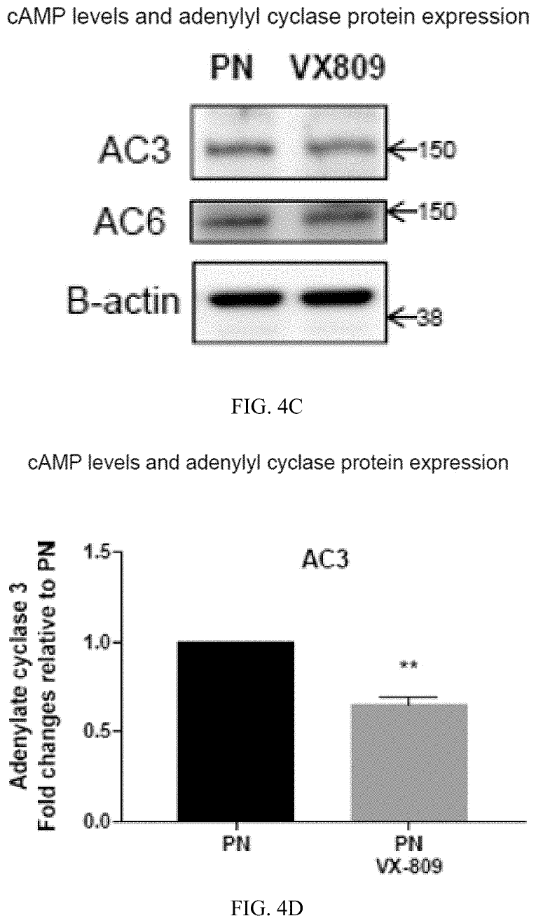

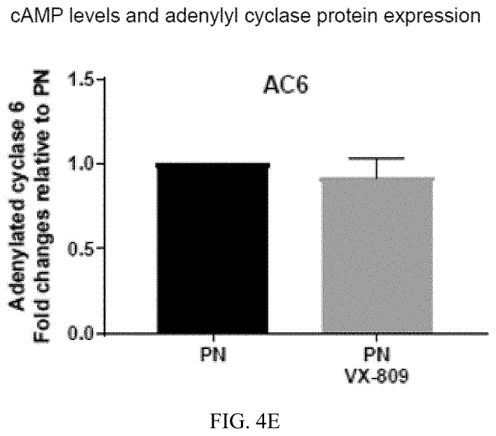

It has been shown previously in both animal and cell culture models of ADPKD that cells lacking functional PC1 have elevated cAMP levels when compared to normal. To assess the effect of CFTR correctors, cAMP activity was measured in PC1 conditional knockout mouse kidneys and in PN cells that were either left untreated or treated with VX-809. Administration of VX-809 (FIG. 4A-B) significantly decreased the cAMP levels. FIG. 4B shows that PH, pkd1 heterozygote, cells have lower resting cAMP compared to PN cells showed previously. Cells were treated with forskolin, which increased cAMP levels drastically (FIG. 4B). It should be noted that VX-809 reduced the forskolin-induced increase in cAMP, indicating a direct action of VX-809 on adenylyl cyclase activity. To determine whether VX-809 also inhibits phosphodiesterase activity, cells were treated with forskolin or with forskolin in combination with 3-isobutyl-1-methylxanthine (IBMX), a phosphodiesterase inhibitor, to maximally stimulate adenylyl cyclase activity. Treating PN cells with forskolin or IBMX (FIG. 4B) increased the cAMP activity by approximately 10-fold. However, VX-809 did not affect the forskolin- or IBMX-induced increases in adenylyl cyclase activity (compare data bars 5 and 9, FIG. 4B), indicating that VX-809 does not affect phosphodiesterase activity. These data suggest that under basal conditions, one way that VX-809 reduces cyst growth (as shown in FIG. 1) is most likely by reducing the resting cAMP levels. However, the results presented above, indicating that VX-809 can inhibit cyst growth even in the presence of forskolin (which elevates cAMP activity almost 500-fold), strongly suggest that other mechanisms are also involved.

### Example 5

VX-809 Regulates AC3 but not AC6

Ca²⁺-dependentadenylyl cyclase activity has been shown to play a role in cyst growth in ADPKD (26). There are two classes of adenylyl cyclases that are regulated by intracellular Ca²⁺: one class is activated, and the other inhibited. One member from each of these two classes was focused on: AC3 and AC6. AC6's activity is inhibited by Ca²⁺, whereas that of AC3 is enhanced by increases in intracellular Ca²⁺. Both AC3 and AC6 are expressed in the proximal tubules of rat kidneys, and AC6 is already known to play a role in ADPKD. It is shown (FIG. 4C-E) that AC3 and AC6 are both expressed in PN cells. The AC3 levels were decreased following treatment of the PN cells with VX-809. This result is consistent with the observation that VX-809 reduces both basal and forskolin-stimulated cAMP levels.

### Example 6

VX-809 Downregulates Resting Intracellular Ca²⁺ Levels and Release of Ca²⁺ from the ER

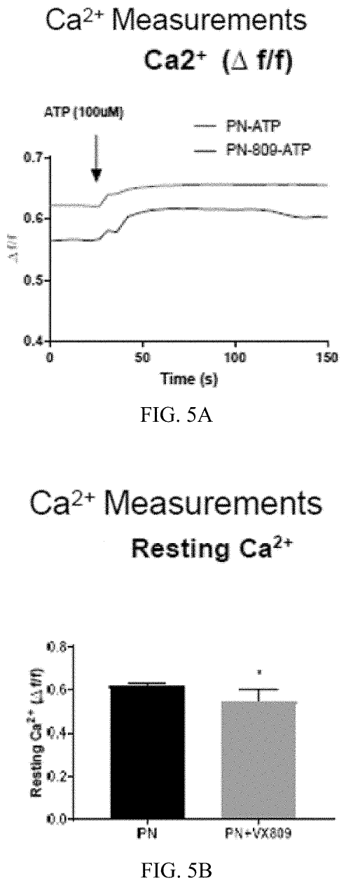

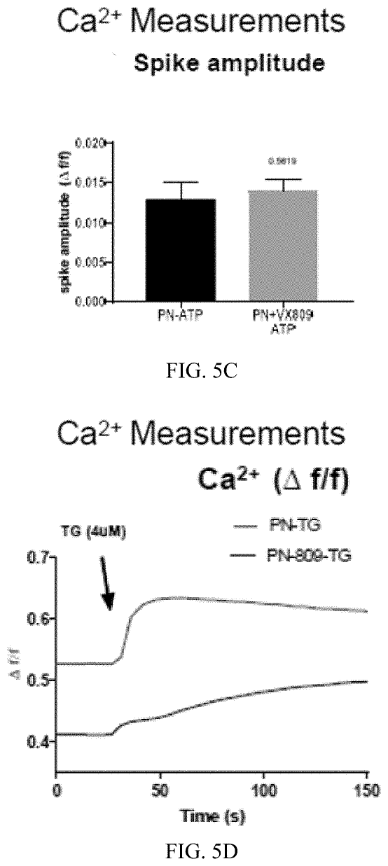

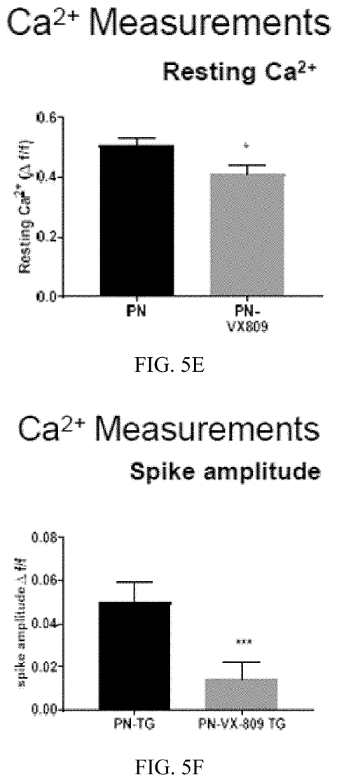

Ca²⁺-dependent signal transduction is associated with cyst formation. To determine whether VX-809 alters Ca²⁺ movement, cells were treated with ATP, which stimulates purinergic receptors, and found (FIG. 5A-C) that VX-809 caused a small reduction in resting Ca²⁺ but had no effect on intracellular Ca²⁺ movement in response to ATP. PC2 was suggested to be a positive regulator of ER Ca²⁺. To address the effect of VX-809 on this Ca²⁺ release, the cells were treated with thapsigargin, a specific inhibitor of the ER Ca²⁺-ATPase which, when applied, allows Ca²⁺ to leak out of the ER through independent Ca²⁺-permeable pathways. A small effect of VX-809 on resting Ca²⁺ was observed. VX-809 dramatically reduced the thapsigargin-induced release of Ca²⁺ from the ER (FIG. 5, D-F).

### Example 7

VX-809 has No Effect on PC2

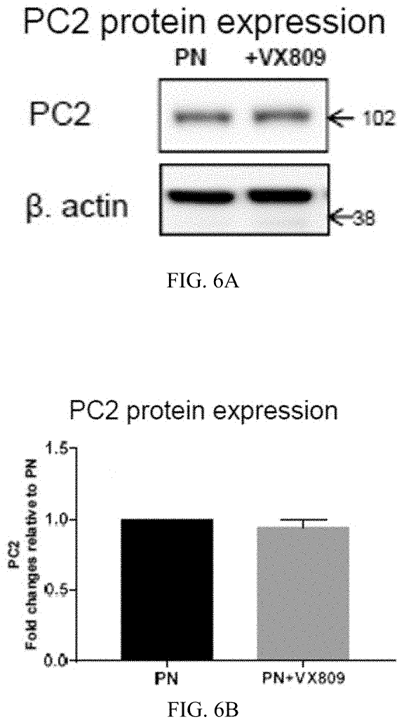

PC2 is a 968-amino acid protein with an approximate molecular weight of 100 kDa in its monomeric form To determine whether VX-809 affects PC2 protein expression, western blot experiments were conducted on PN cells to determine the resting levels of endogenous PC2. The results indicated (FIG. 6) that VX-809 has no effect on PC2 expression.

### Example 8

VX-809 Reduces the Steady-State Levels of Heat Shock Proteins

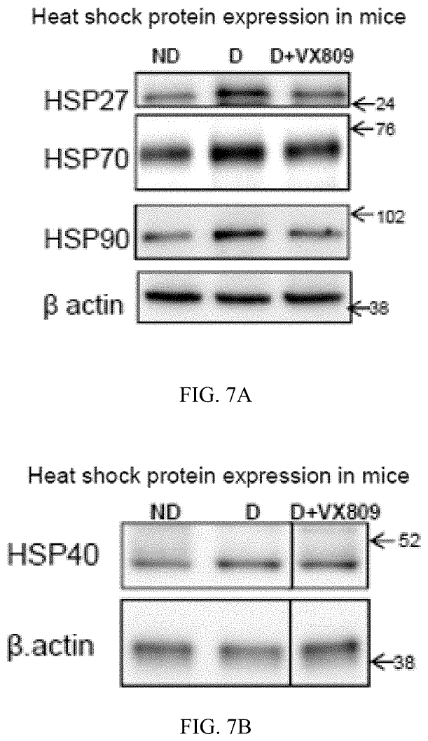

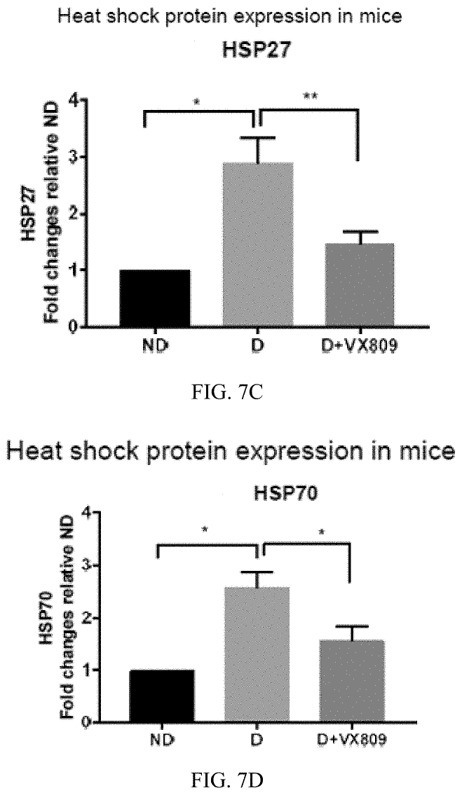

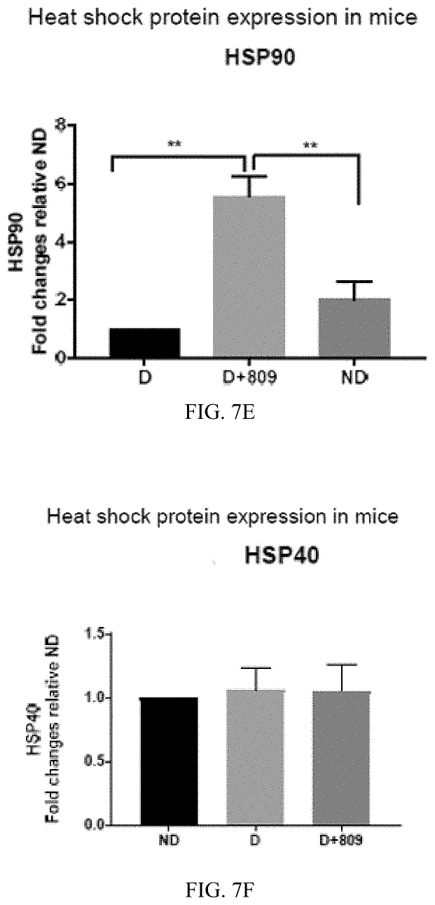

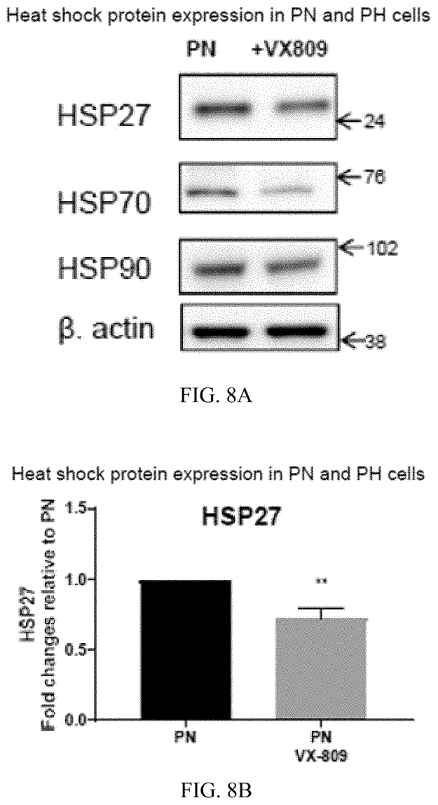

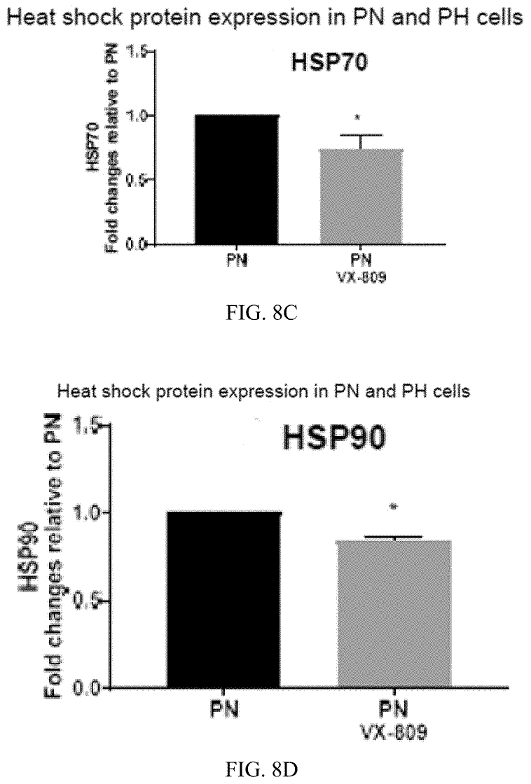

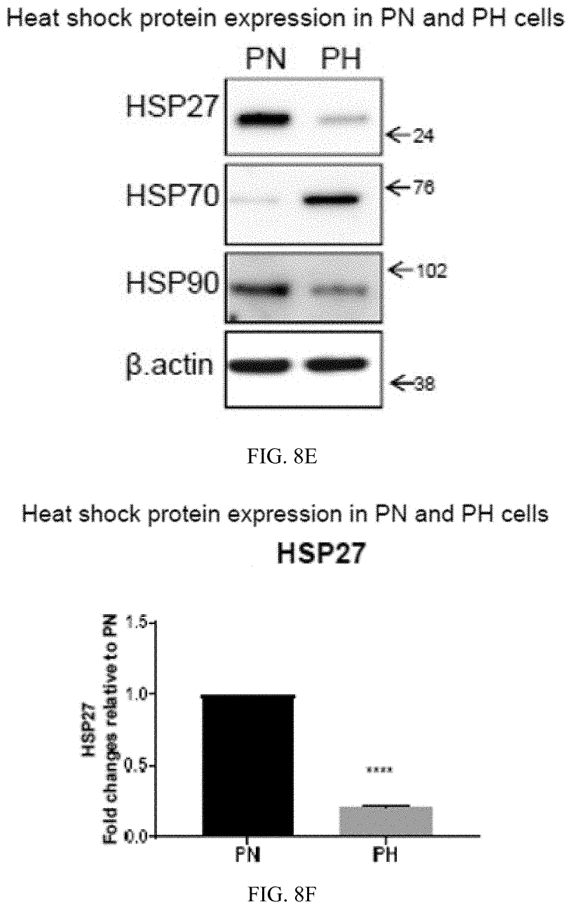

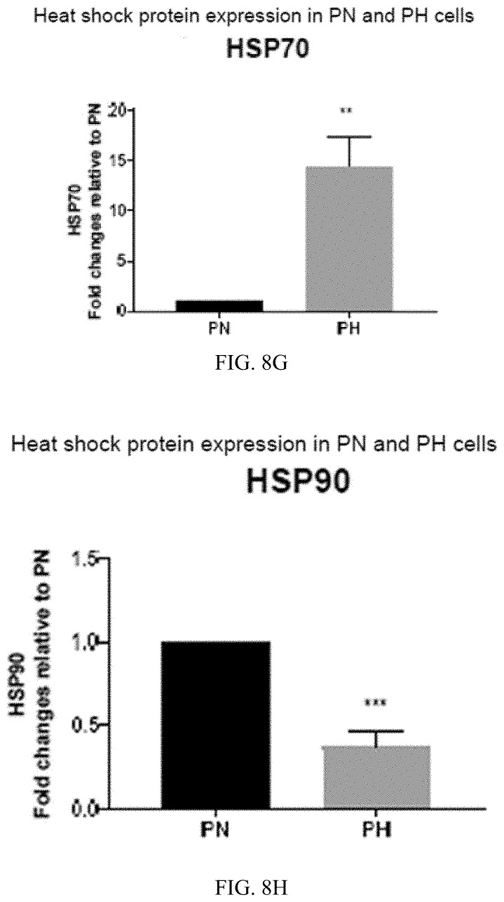

To facilitate their growth in the kidney, cysts have developed an altered network of proteins that most likely serves to protect them from stress . Next, it was determined whether VX-809 would alter the levels of Hsps in pkd−/− mice and in PN cells. FIG. 7 shows the results in the mice. Hsp 27, 70 and 90 are all elevated in cystic kidneys compared to normal controls. Treatment with VX-809 of kidneys in which PC1 levels had been knocked out resulted in significantly less expression of Hsp27, Hsp70, and Hsp90. There is no change in Hsp40. Likewise, PN cells treated with VX-809, FIG. 8A-D showed reduced expression of Hsp27, Hsp70, and Hsp90 following treatment. Both sets of data are consistent with an alteration in the heat shock response to cyst growth. Comparing PH, which are pkd+/− compared to the PN cells which are pkd−/− showed that Hsp27 and 90 are elevated and Hsp70 reduced in the PN vs. the PH cells (FIG. 8F-H).

### Example 9

VX-809 Enhances the Ddisappearance of Hsp70 and Hsp90

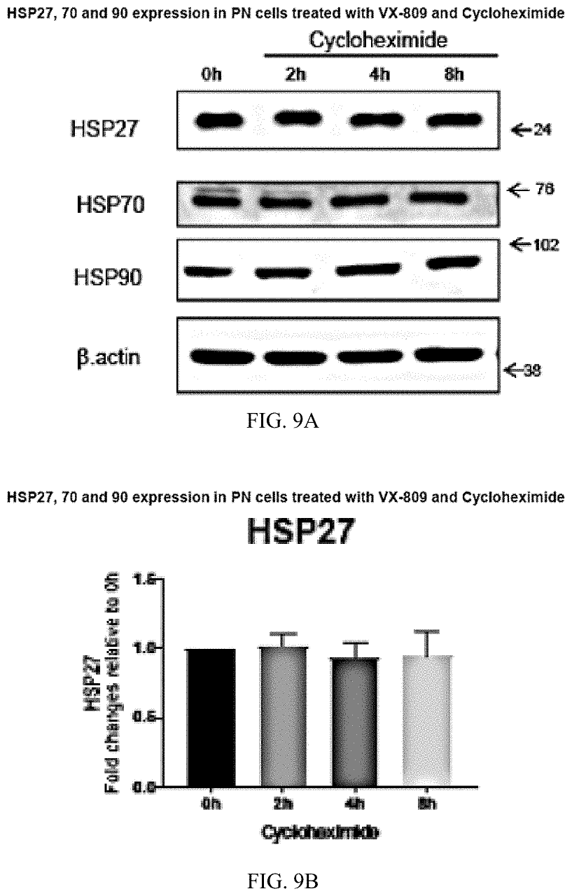

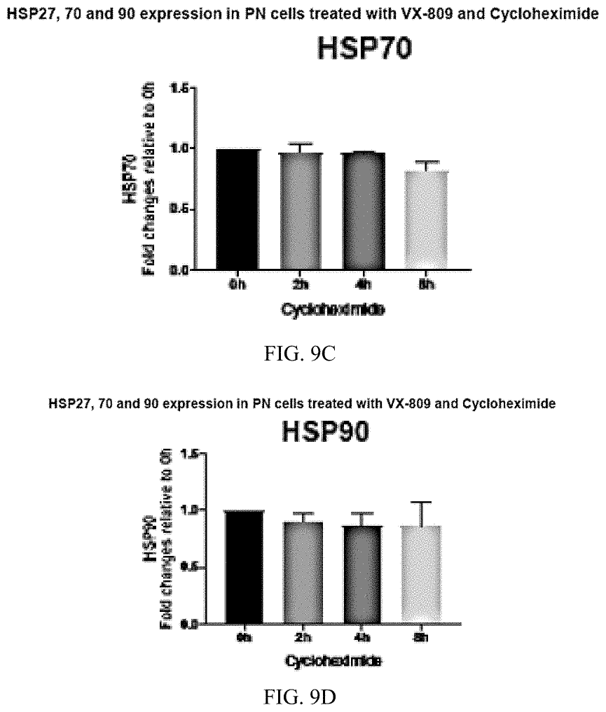

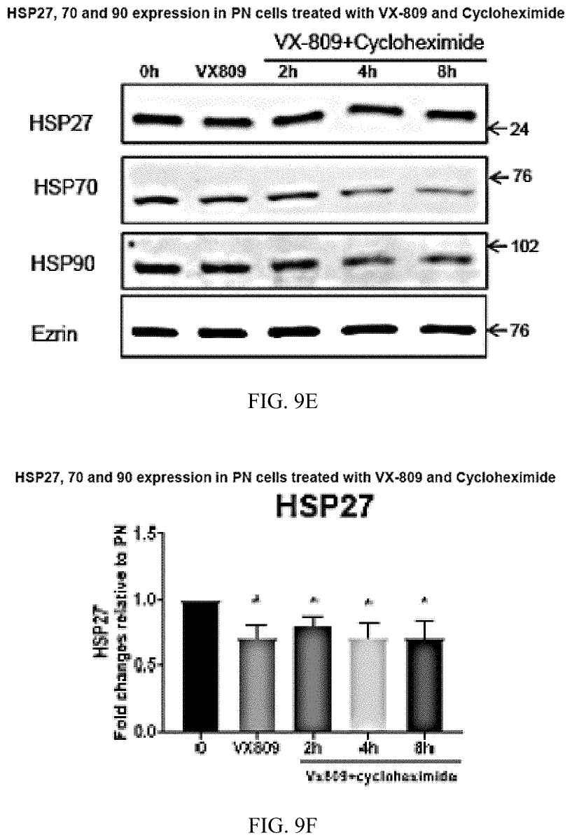

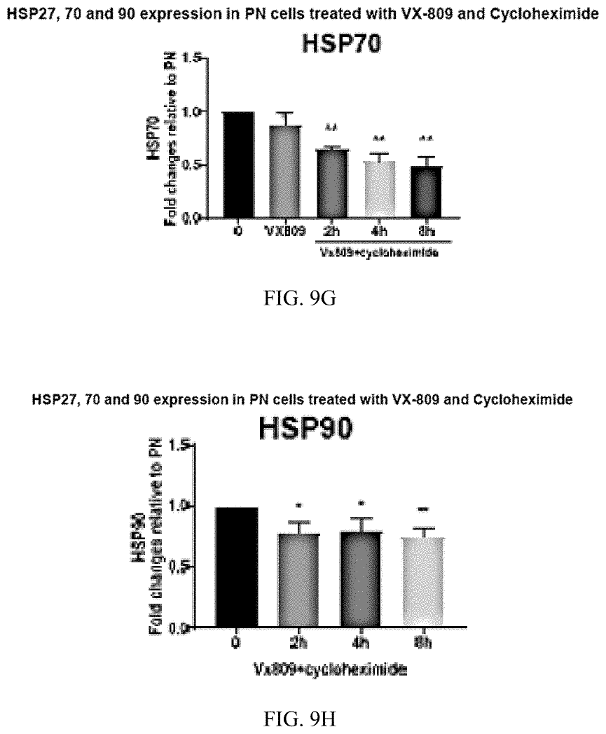

To gain more insight into the mechanism whereby VX-809 alters Hsp protein levels, translation was inhibited by using cycloheximide and the disappearance of the three Hsps was monitored. FIG. 9A-D shows that the steady-state levels of each of the Hsps was unchanged over the 8 h immediately following cycloheximide treatment, suggesting that they are long-lived, stable proteins. In contrast, after treatment with VX-809, the Hsp70 levels dropped over the 8-h period to approximately half the level observed at time 0. Hsp90 was reduced by approximately 25%, and Hsp27 was unchanged. These data suggest that VX-809 reduces the half-life of two key Hsps, most likely by increasing their rates of degradation.

### Example 10

VX-809 Reduces Apoptosis

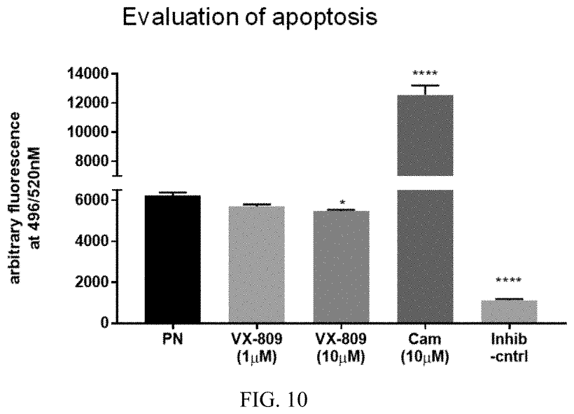

Apoptosis is associated with the absence of PC1 in ADPKD. To assess whether apoptosis is directly altered by VX-809, caspase 3 activity was monitored in PN cells. FIG. 10 shows that there was a small reduction in caspase 3 activity in the PN cells after treatment with VX-809.

### Example 11

VX-809 Dramatically Reduces the ER Stress-Related Protein GADD153

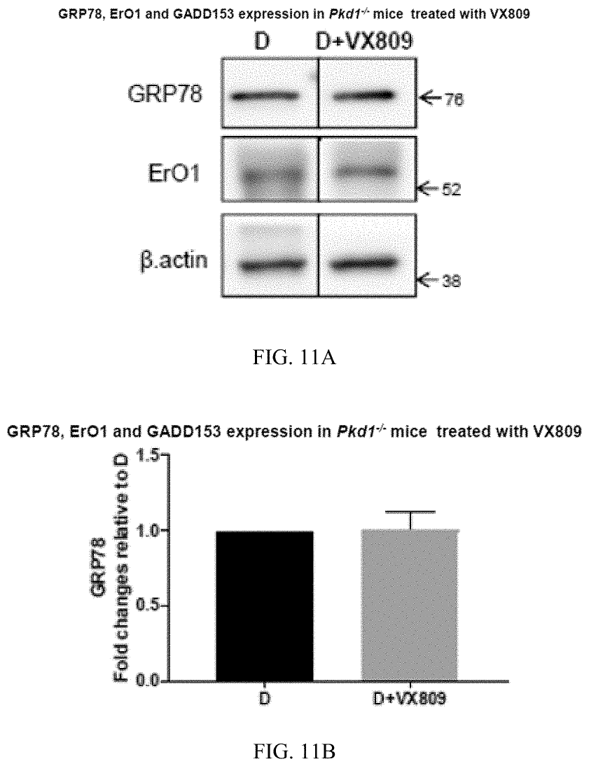

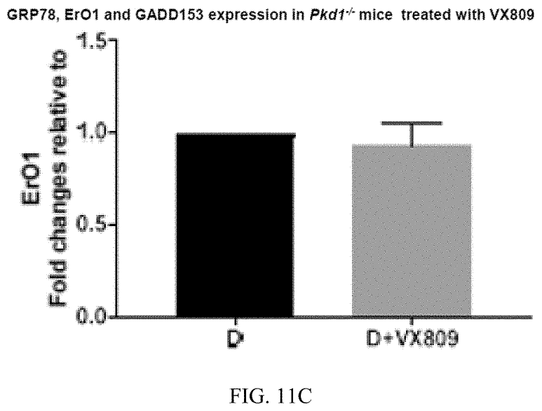

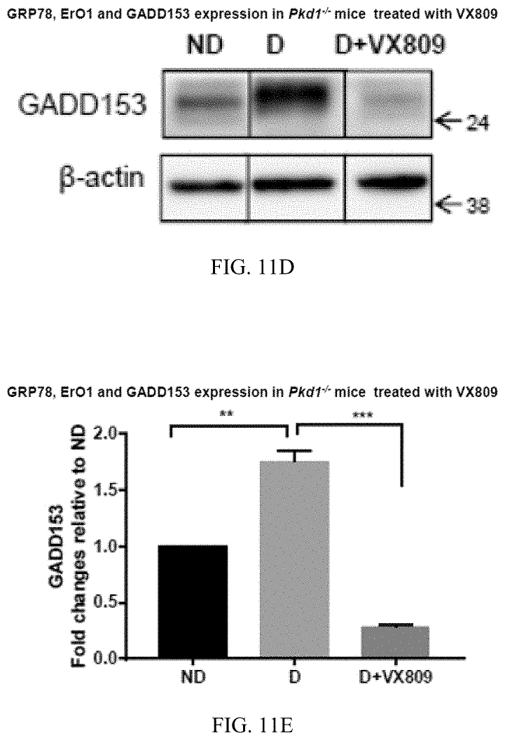

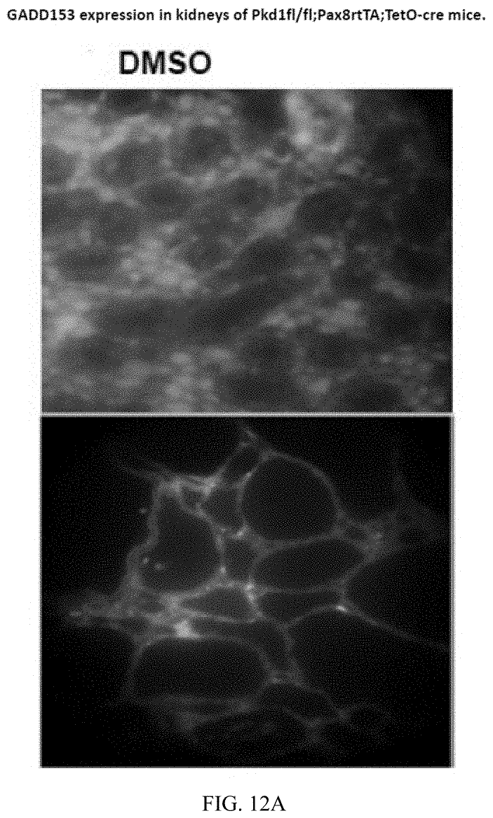

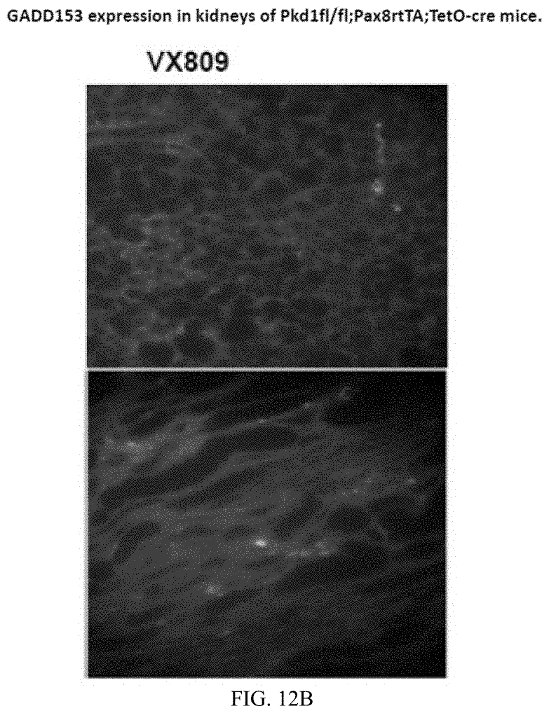

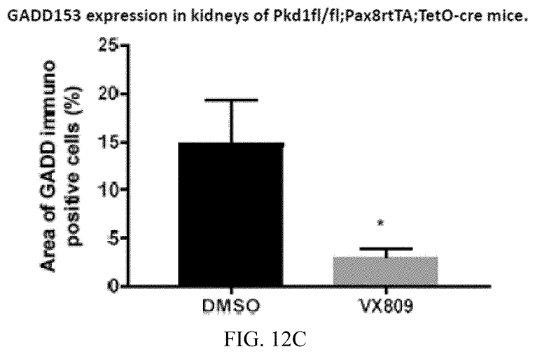

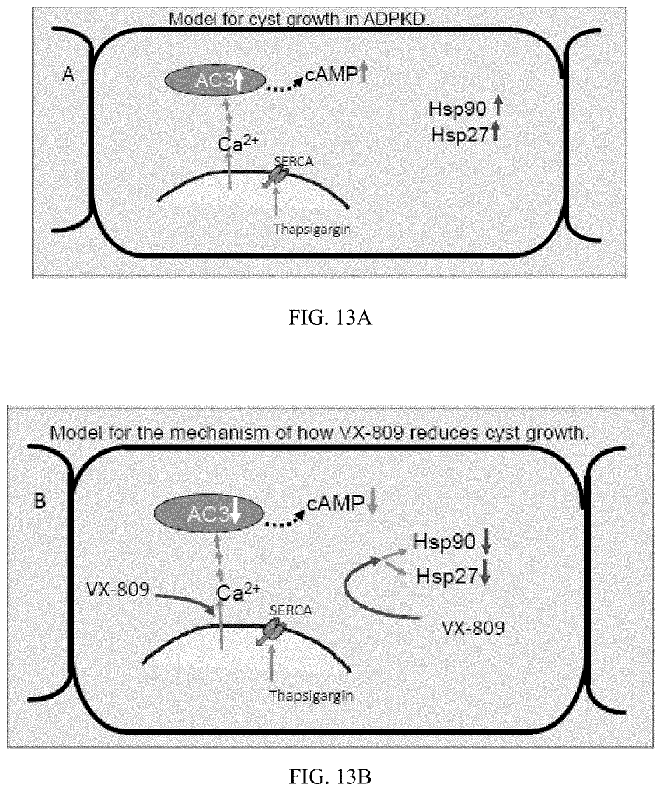

Given that VX-809 alters an ensemble of Hsps, it was evaluated whether VX-809 alters proteins associated with ER stress. To address this question, the levels of three ER stress-associated proteins were measured, 78-kDa glucose-regulated protein (GRP78), ER oxidoreductin 1 (Ero1) and DNA damage-inducible protein 3 (GADD153), also known as C/EBP homologous protein (CHOP) (37). The results are depicted in FIG. 11 show no changes in GRP78 or Ero1 in response to VX-809. In sharp contrast, there is a dramatic increase in GADD153 when cysts are induced in the mice compared to their normal littermates and an equally dramatic reduction in GADD153 reduction when the cyst containing mice are treated with VX-809. Immunostaining for GADD153 in the mice kidneys also shows a strong reduction when mice are treated with VX-809 (FIG.12).

### Example 12

NHE3 Expression and Activity

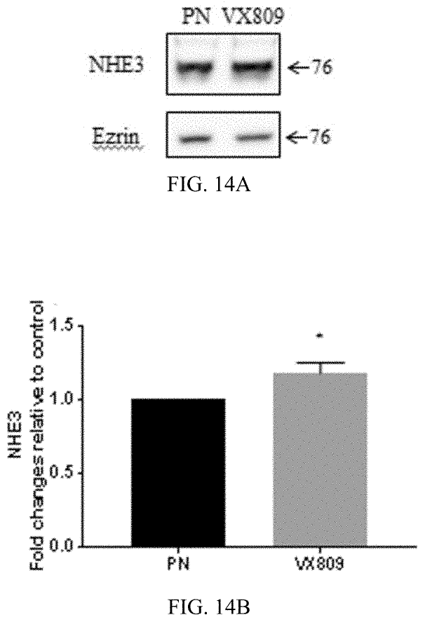

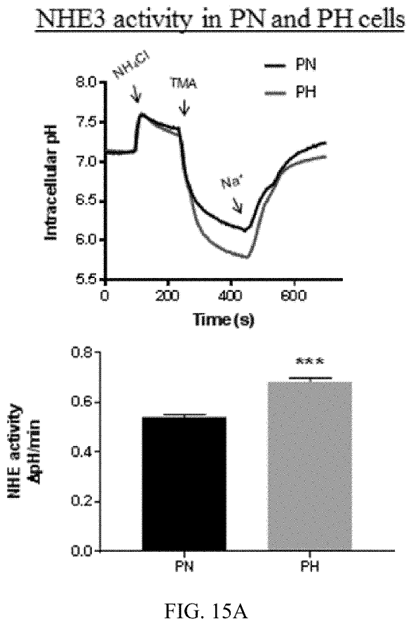

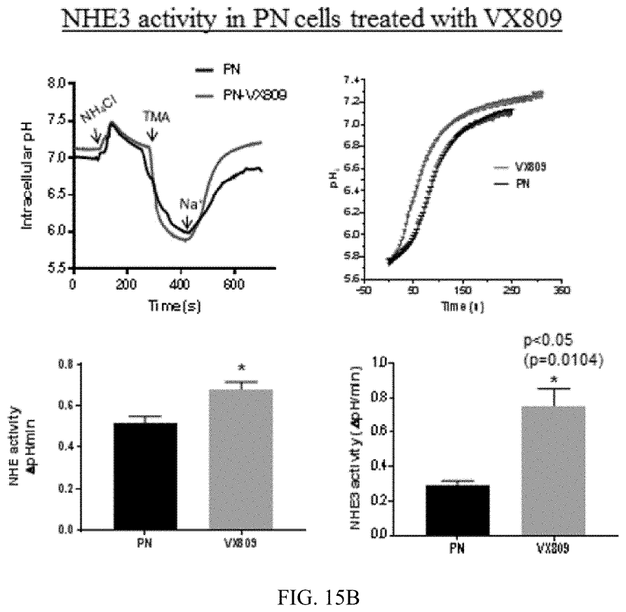

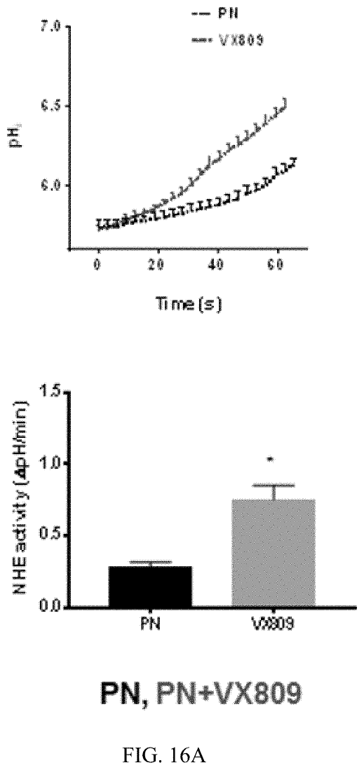

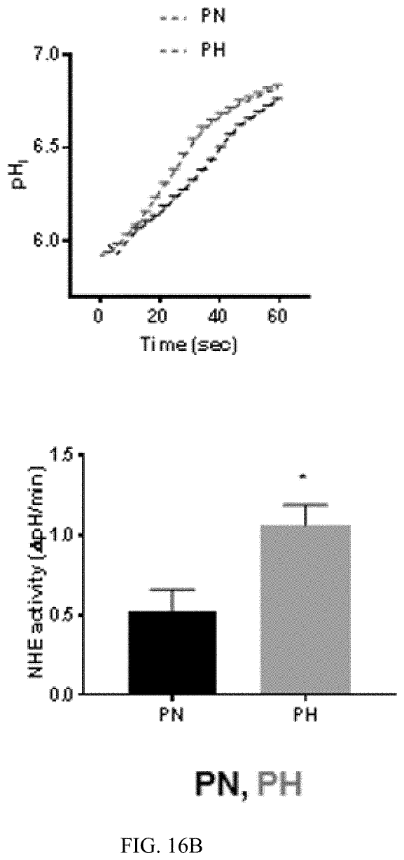

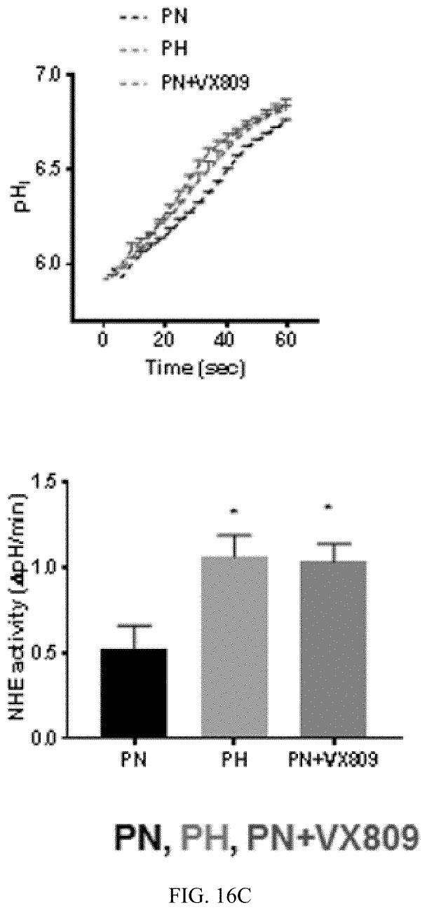

The expression and activity of NH3 were evaluated. Specifically, NHE3 expression was evaluated in PN cells that were treated with VX-809. This study shows that NHE3 expression is significantly increased in PN cells treated with VX-809, as compared with PN cells (FIG. 14A and FIG. 14B). NH3 expression was also studied in PN, PH, and PN cells treated with VX-809. It was found that NHE3 activity is significantly reduced in PN cells, as compared with the control PH cells (FIG. 15A). Further, NHE3 activity is significantly increased in PN cells treated with VX-809, as compared with PN cells (FIG. 15B). Next, NHE3 activity was evaluated in PN/PH cells or PN cells treated with VX-809. NHE3 activity was increased in PN cells treated with VX-809, as compared to PN cells (FIG. 16A). NHE3 activity was higher in control PH cells than in PN cells (FIG. 16B). Treatment of PN cells with VX-809 led to similar activity levels of NHE, as compared to NHE3 activity in control PH cells (FIG. 16C). NHE3 activity was increased in both VX-809 treated PN cells and control PH cells, as compared to control PN cells (FIG. 16C).

### Example 13

Localization Studies of PC2 and CFTR

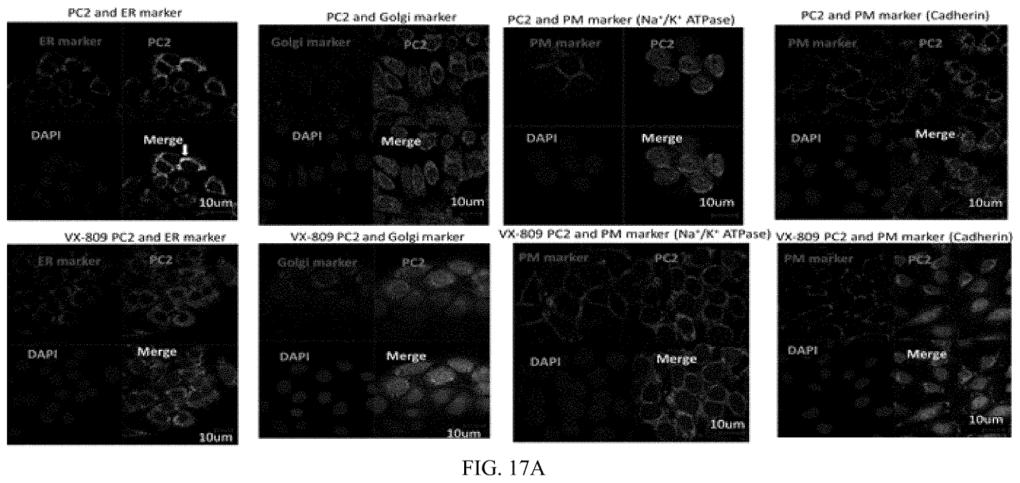

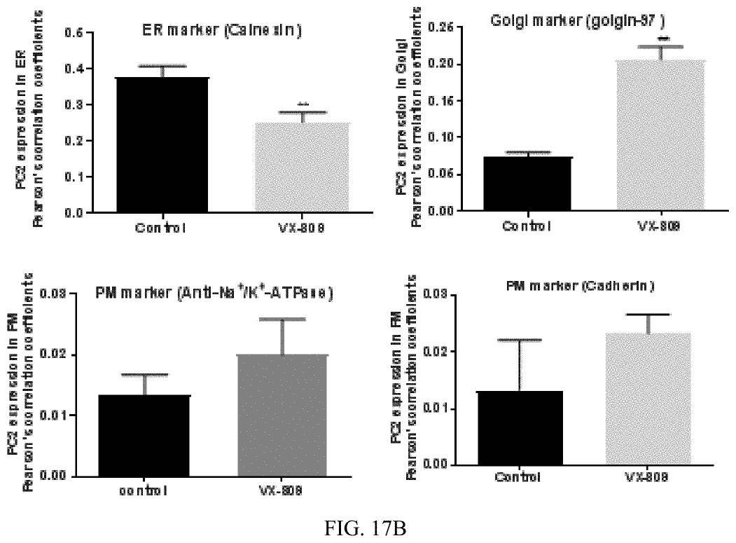

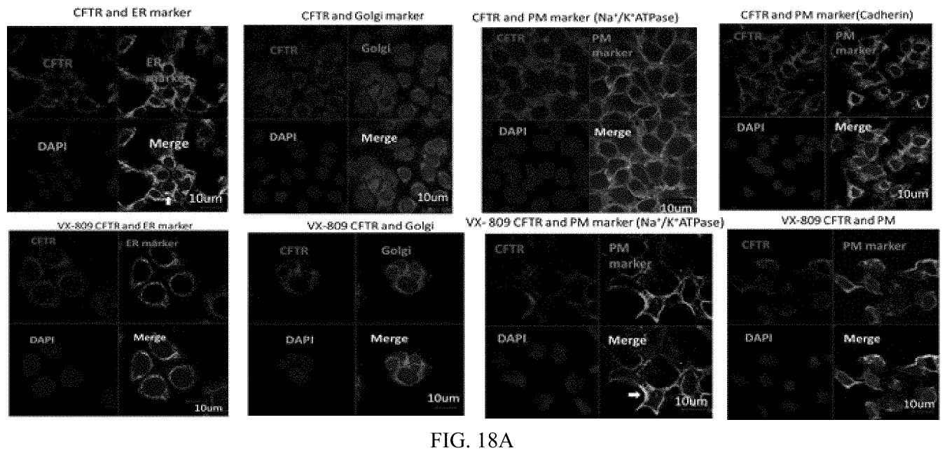

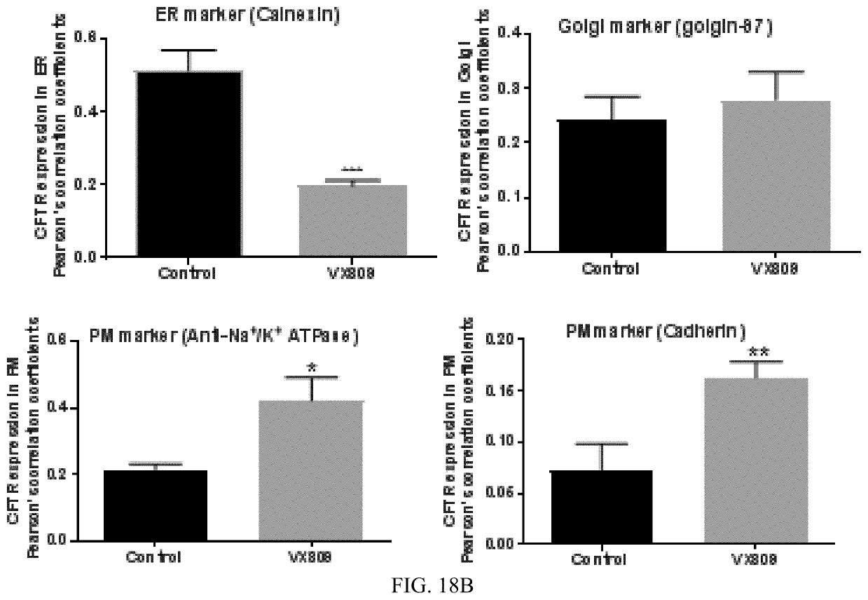

The localization of PC2 and CFTR in control cells was compared to the localization of PC2 and CFTR localization in VX-809 treated cells. The evaluation of PC2 and an ER marker, PC2 and a Golgi marker, PC2 and a PM marker (NA+/K+ ATPase), and PC2 and a PM marker (cadherin) show that with the treatment of VX-809, there is a statistically significant move of PC2 from the ER to Golgi (FIG. 17A and FIG. 17B). The evaluation of CFTR and an ER marker, CFTR and a Golgi marker, CFTR and a PM marker (NA+/K+ ATPase), and CFTR and a PM marker (cadherin) show that with the treatment of VX-809, there is a statistically significant move of CFTR from the ER to Basolateral and Apical Membrane (FIG. 18A and FIG. 18B).

### Example 14

CFTR Expression in PN Cells

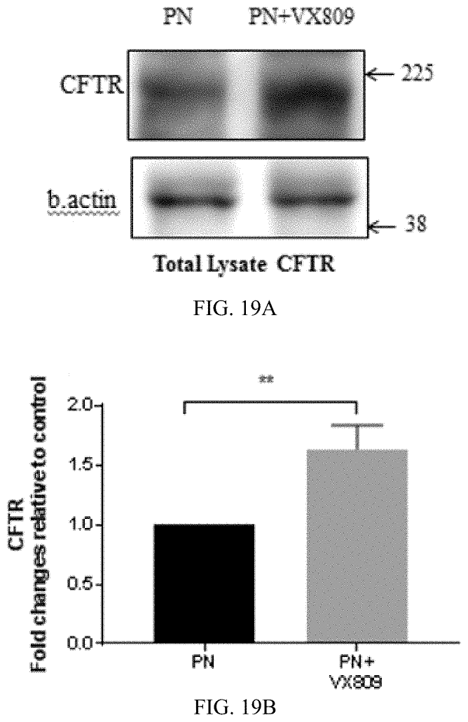

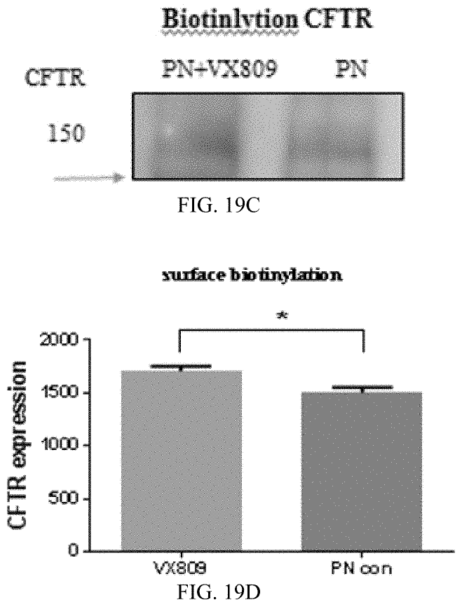

The expression of CFTR was evaluated in PN cells treated with VX-809 and control PN cells. PN cells were treated with 10 μM VX-809 for 16 h or a control. VX-809 treatment enhanced CFTR expression, as compared with control PN cells (FIG. 19A and FIG. 19B). The biotinylation of CFTR was significantly increased in PN cells treated with VX-809 treatment, as compared to control PN cells (FIG. 19C and FIG. 19D).

The examples disclosed herein show at least that: 1) CFTR moves out from the ER to Basolateral and Apical Membranes; 2) PC2 Moves out from the ER to the Golgi; 3) Heat Shock Proteins, Hsp27, 70, 90 are reduced; 4) Na reabsorption is restored; 5) Ca release from ER is reduced; 6) cAMP levels are reduced by reducing AC3, 7) Cyst growth in PT, DT and Collecting duct is reduced; and 8) AQP2 is restored in the collecting duct.

### Example 15

Experimental Methods

Cell culture and reagents. Pkd1-null (PN) and-heterozygous (PH) cells were cultured as previously described. Forskolin (#11018) was purchased from Sigma (SC23950); VX-809 (#S1565) was purchased from Selleck chemicals, Huston, Tex., USA; Ezrin (SC58758) adenylate cyclase 3 (SC588), PC2 (SC28331), Hsp27 (SC13132), Hsp70 (SC66048), anti-GADD153 antibody (SC7351), anti-Ero1 antibody (SC365526), and P-actin (SC47778) were purchased from Santa Cruz Biotech, Tex., USA Hsp90 (ADI-SPA-830F) was purchased from Enzo Life Sciences, N.Y., USA AC6 (GTX47798) was purchased from GeneTex, Irvine, Calif., USA Anti-GRP78 BiP antibody was purchased from Abeam (Cat#ab21685).

Mouse strain and treatment. Pkd1^{fl/fl}; Pax8^{rtTA}; TetO-cre mice on a C57BL/6 background (61) were provided by the Baltimore PKD Center. Mice of both sexes were used in this study. Mice were injected IP with doxycycline (Sigma, #D9891) (4 μg of doxycycline/g body weight) on postnatal day 11 (PND11), PND12, and PND13 to produce very rapid and aggressive cyst growth. On PND21, the mice were euthanized.

In vitro cytogenesis. To induce differentiation, cells were kept at 37° C. for at least 6 days without gamma-interferon. After one week, the cells were used for 3D culture or other experiments. Growth factor-reduced Matrigel #354230 (Corning) was used, and cell dilutions were prepared so that there were approximately 6,000 cells in 25 μl of medium; 25 μl of cell preparation was mixed with 50 μl of Matrigel. Pictures were taken with a Zeiss Axio microscope. Cystic areas were analyzed with ImageJ (provided by the NIH).

Cyclic AMP assay. Confluent cells were treated with VX-809 (10 μM) or DMSO for 16 h before being harvested for assay. Cyclic AMP levels were measured with a direct cAMP Enzyme Immunoassay Kit (Sigma, #CA200) according to the manufacturer's protocol. The results are expressed as pmole/ml. Statistical analysis was performed using a two-tailed Student's t-test.

Fura-2 Ca²⁺ imaging assay. On Day 5 of cell culture, the cells were loaded with the cell-permeant acetoxymethyl (AM) ester of the calcium indicator fura-2 (fura-2/AM) at 37° C. for 90 min. Measurements were made on a Zeiss inverted microscope equipped with a Sutter Lambda 10-2 controller and filter wheel assembly. For ATP stimulation experiments, the cells were exposed to 100 μM ATP diluted in the imaging buffer. A Zeiss FluorArc mercury lamp was used to excite the cells at 340 and 380 nm, and the emission response was measured at 510 nm Cell fluorescence was measured in response to excitation for 1000 ms at 340 nm and 200 ms at 380 nm once every 4 s. Image acquisition, image analysis, and filter wheel control were performed with IPLab software.

### Example 16

To determine the location of PC2 in the cilia, confocal microscopy experiments using the Pkd1^{fl/fl}; Pax8^{rtTA} TetO-cre mouse model were conducted. These mice, when injected intraperitoneally (IP) with doxycycline on postnatal days (PND) 11, 12, and 13, developed multiple large cysts and large polycystic kidneys at approximately 3 weeks of age. Cystic mice injected daily with VX-809, when compared to DMSO-treated mice from PND10 to PND20, show significantly less cyst growth and an improved renal function.

FIG. 20 shows confocal microscopic images of stained kidney sections in Pkdl mice. Mice were treated without doxycycline (NoDoxy), with doxycycline plus DMSO (Doxy), or with doxycycline plus VX809 (Doxy VX809), as indicated. PC2 staining is shown in red, acetylated (ac) αtubulin is shown in green, regions of colocalization of PC2 and ac-atubulin are shown in yellow. Scale bar is 10 μm. All pictures were taken with a 63× objective.

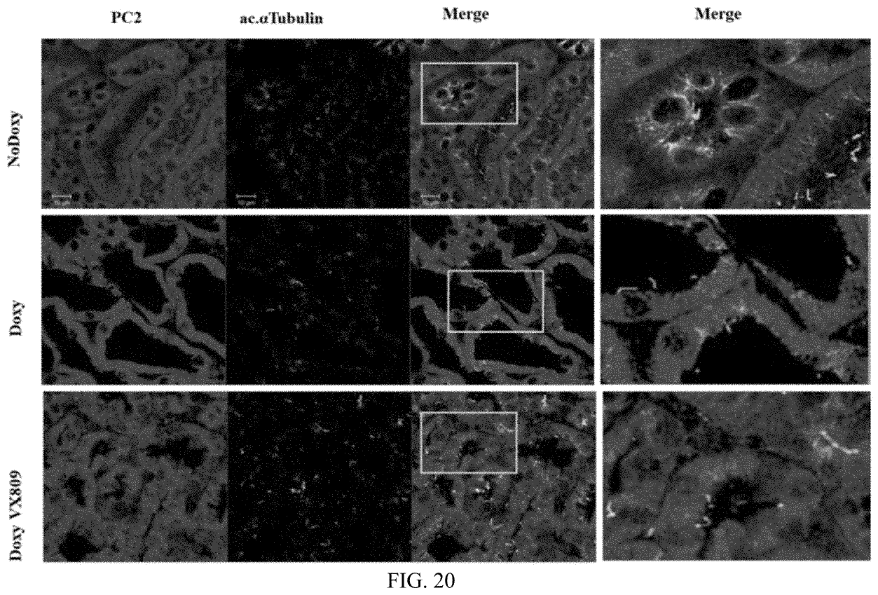

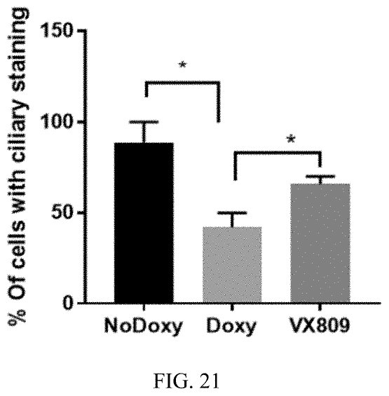

FIG. 21 is a graph of the percentage of cells with ciliary staining. Mice were treated without doxycycline (NoDoxy), with doxycycline plus DMSO (Doxy), or with doxycycline plus VX809 (Doxy VX809), as indicated, and kidney cells were analyzed. Pearson's coefficient was measured using at least 10 cells per image. Three animals were analyzed for each maneuver.

In normal mice PC2 is located in the primary cilium. When cysts are induced to form with doxycycline, however, PC2 is drastically reduced in the cilium. Treatment of mice with VX-809 increases colocalization of PC2 with the cilia marker ac-atubulin and restores PC2 to the cilium in response to doxycycline. The results suggest that restoration of PC2 to the primary cilium is a critical aspect of how CFTR modulators reduce cyst growth.

The levels of PC2 protein were analyzed in two mouse models of ADPKD: Pkd1^{fl/fl}; Pax8^{rtTA}; TetO-cre, which is PC1 null, and the Pkd1^{R3277C/R3277C} (pkd1^{RC/RC}) which has a missense mutation in PC1. Pkd1^{RC/RC} mice develop a consistent, slowly progressing polycystic kidney disease. Pkd1^{RC/RC} mice were treated daily with VX-809, beginning at one month, and euthanized at 6 months.

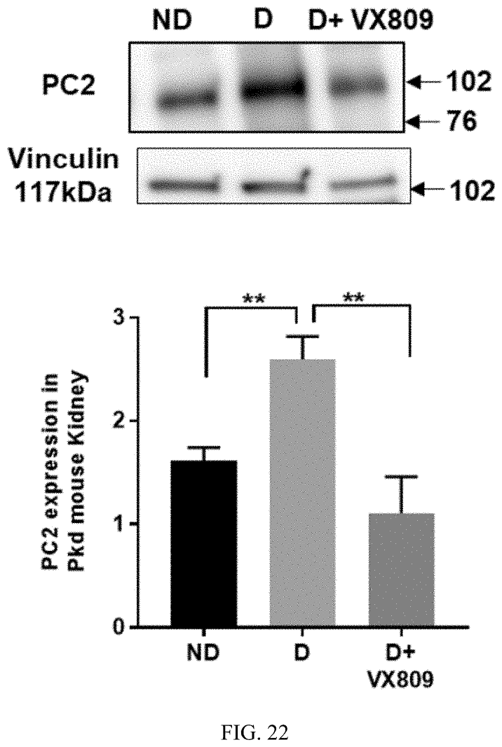

FIG. 22 shows an immunoblot and graph of PC2 protein levels in kidney tissue of Pkd1^{fl/fl}; Pax8^{rtTA}; TetO-cre mice. The upper panel shows immunoblots of PC2 and vinculin, which was used as control for protein levels. The lower panel shows a graph of PC2 expression normalized to vinculin levels. Mice were treated without doxycycline (ND), with doxycycline plus DMSO (D), or with doxycycline plus VX809 (D+VX809), as indicated. Three mice were analyzed for each treatment.

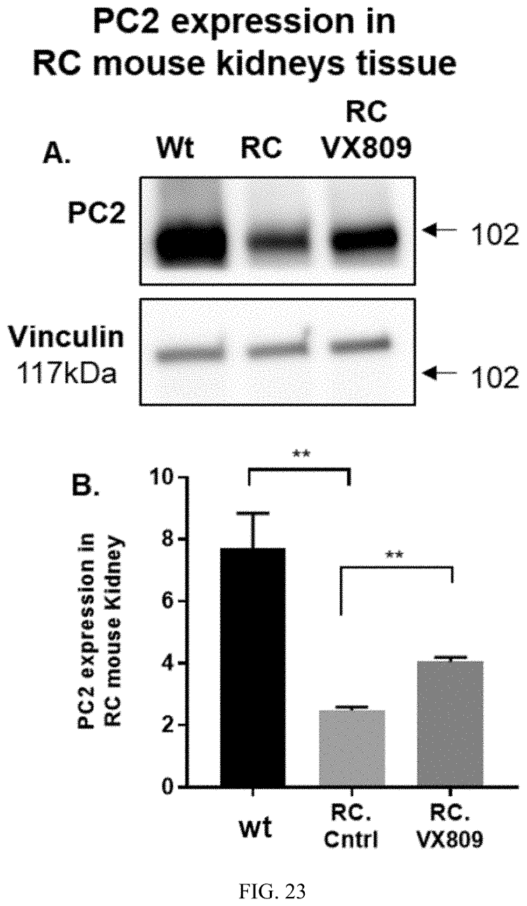

FIG. 23 shows an immunoblot and graph of PC2 protein levels in kidney tissue of wild-type and Pkd1^{RC/RC} mice. The upper panel shows immunoblots of PC2 and vinculin, which was used as control for protein levels. The lower panel shows a graph of PC2 expression normalized to vinculin levels. Wild-type mice were treated without doxycycline (Wt), and Pkd1^{RC/RC} mice were treated with doxycycline plus DMSO (RC) or with doxycycline plus VX809 (RC+VX809), as indicated. Three mice were analyzed for each treatment.

In the Pkd1^{fl/fl} mice, doxycycline-induced cyst formation increases PC2 protein. In these mice, VX-809 mitigates the doxycycline-induced increase in PC2 and restores PC2 expression to near-normal values.

In Pkd1^{RC/RC} mice, doxycycline-induced cyst formation decreases PC2 protein. In these mice, VX-809 mitigates the doxycycline-induced decrease in PC2, resulting in higher levels of PC2 expression.

Taken together, the results in Example 16 support the idea that VX-809 reduces cyst growth by altering the location and expression of PC2.

Various changes and modifications to the disclosed embodiments may be apparent to those skilled in the art. Such changes and modifications, including without limitation those relating to the chemical structures, substituents, derivatives, intermediates, syntheses, compositions, formulations, or methods of use of the invention, may be made without departing from the spirit and scope thereof.

### INCORPORATION BY REFERENCE

References and citations to other documents, such as patents, patent applications, patent publications, journals, books, papers, web contents, have been made throughout this disclosure. All such documents are hereby incorporated herein by reference in their entirety for all purposes.

### Equivalents

Various modifications of the invention and many further embodiments thereof, in addition to those shown and described herein, will become apparent to those skilled in the art from the full contents of this document, including references to the scientific and patent literature cited herein. The subject matter herein contains important information, exemplification, and guidance that can be adapted to the practice of this invention in its various embodiments and equivalents thereof.

What is claimed is:

1. A method of treating polycystic kidney disease in a subject, the method comprising providing to a subject that has a form of CFTR that is not associated with cystic fibrosis an agent that increases PC2 in a cilium of a cell in order to lower calcium ion signaling in cystic cells and reduce their growth, thereby treating polycystic kidney disease in the subj ect.

2. The method of claim 1, wherein the increase in PC2 in the cilium of the cell is associated with one selected from the group consisting of increased expression of PC2 and increased delivery of PC2 to the cilium.

3. The method of claim 1, wherein the agent is a CFTR modulator.

4. The method of claim 3, wherein the CFTR modulator is a CFTR corrector.

5. The method of claim 4, wherein the CFTR corrector is lumacaftor.

6. The method of claim 1, wherein the condition is autosomal dominant PKD (ADPKD).

7. The method of claim 1, wherein the agent also increases PC1 in the cilium of a cell

8. The method of claim 7, wherein the increase in PC1 in the cilium of the cell is associated with one selected from the group consisting of increased expression of PC1 and increased delivery of PC1 to the cilium.

9. The method of claim 7, wherein the CFTR modulator is a CFTR corrector.

10. The method of claim 9, wherein the CFTR corrector is lumacaftor.
